(19) United States
(12) Patent Application Publication
(10) Pub. No.: US 2023/0241017 A1
(43) Pub. Date: Aug. 3, 2023

(54) Hydrophilic Degradable Microsphere for Delivering Travoprost

(71) Applicants: Occlugel, Les Ulis (FR); Centre National de la Recherche Scientifique (CNRS), Paris (FR); Université Paris-Saclay, Gif Sur Yvette (FR)

(72) Inventors: Anne Beilvert, Massy (FR); Emeline Corus, Janvry (FR); Laurent Bedouet, Les Mans (FR); Laurence Moine, Saint Cloud (FR)

(21) Appl. No.: 18/003,180

(22) PCT Filed: Jul. 7, 2021
(86) PCT No.: PCT/EP2021/068911

§ 371 Date: Dec. 23, 2022

(30) Foreign Application Priority Data
Jul. 7, 2020 (EP) 20305777.3

(51) Int. Cl. A61K 31/216; A61K 9/16

(52) U.S. Cl. CPC A61K 31/216; A61K 9/1635; A61K 9/1641

(57) ABSTRACT

The invention relates to a composition comprising an effective amount of a prostaglandin analogue, at least one hydrophilic degradable microsphere comprising a crosslinked matrix, and a pharmaceutically acceptable carrier for administration by injection, the crosslinked matrix being based on at least a) between 10 mol % and 90 mol % of hydrophilic monomer of general formula (I); b) between 0.1 and 30 mol % of a cyclic monomer of formula (II); and c) between 5% and 90% of one degradable block copolymer cross-linker, wherein the degradable block copolymer crosslinker is linear or star-shaped and presents (CH₂═(CR₁₁))-groups at all its extremities. The invention also relates to such a composition for use for preventing and/or treating moderate to ocular hypertension or glaucoma.

### FIELD OF THE INVENTION

The present invention relates to hydrophilic degradable microspheres for delivering a prostaglandin analogue. In particular, the present invention relates to composition comprising an effective amount of prostaglandin analogues and hydrophilic degradable microspheres. The present invention also relates to said composition for use for preventing and/or treating ocular hypertension or glaucoma.

### TECHNICAL BACKGROUND

Glaucoma is a disease that damages the eye's optic nerve leading to progressive, irreversible vision loss. Glaucoma usually happens when fluid builds up in the front part of the eye. That extra fluid increases the pressure in the eye, inducing cell death of retinal ganglion cell neurons and their axons damaging the optic nerve over time. The damages created are irreversible, glaucoma is the second cause of blindness in the world. Actually around 60 million people are affected worldwide while 100 million are forecast in 2040 (Yadav 2019, Materials Science & Engineering C: 103). The economic burden of glaucoma is important, with an overall cost to Medicare of $748 million in 2009 (Lambert 2015, Transl Vis Sci Technol. 4(1)). Annual eye care-related costs for glaucoma patients with no vision loss were $8157 (2007 US dollars); this increased to $14,237 for moderate to severe vision loss before reaching $18,670 for patients blinded by the disease.

The two main types of glaucoma are primary open-angle glaucoma and angle-closure glaucoma. Primary open-angle glaucoma is the most common form of the disease. The drainage angle formed by the cornea and iris remains open, but the trabecular meshwork is partially blocked. This leads to a gradual increase in pressure in the eye. This pressure damages the optic nerve and may lead to vision loss without signs or symptoms.

Angle-closure glaucoma, also called closed-angle glaucoma, occurs when the iris swells forward to narrow or block the drainage angle formed by the cornea and iris. As a result, fluid cannot circulate through the eye and pressure increases. Angle-closure glaucoma may occur suddenly (acute angle-closure glaucoma) or gradually (chronic angle-closure glaucoma).

If treated early, the progression of glaucoma can be slowed or stopped with medication, laser treatment, or surgery. The goal of these treatments is to reduce intraocular pressure, since it is impossible to repair the optic nerve. Eye drops are the treatment of choice for primary open-angle glaucoma. Treatments are therefore aimed to increase the elimination of aqueous humor, decrease its production or both. Reduction of aqueous humor production is the therapeutic goal achieved with carbonic anhydrase inhibitors (Brinzolamide, Dorzolamide) and B-blockers (Timolol, Levobunolol, Betaxolol, Carteolol). Other medications accelerate its elimination such as prostaglandin analogues (Latanoprost, Bimatoprost, Travoprost).

However, problems with patient adherence to topical medications hinder glaucoma therapy with eyes drops. Regular application of the treatment is essential, as it is assumed that 1 mm Hg decrease in intraocular pressure can reduce the progression of glaucoma by 10%. Discontinuation of treatment after 6 months is reported in 50% of patients (Nordstrom et al; 2005. Am J Ophthalmol. 140:598-606) while the persistence rate of patients drops to 22.5% after one year and 11.5% three years after the first prescription (Quek et al; 2011. Arch Ophthalmol. 129:643-8). Further, a number of patients (13%) fail to use eye drops correctly (Brown et al; 1984. Can J Ophthalmol. 19:2-5). A poor adherence to treatment induces glaucoma progression in 50% of patients (Lambert 2015, Transl Vis Sci Technol. 4(1)).

Non-adherence to glaucoma medications via eye drops may be a major cause of treatment failure. Novel therapeutic solutions need to be developed to allow the daily application of anti-glaucomatous drugs. One of them is the subconjunctival injection of drug delivery systems (DDS) for sustained local delivery. One study in Singapore found that 74% of glaucoma patients were willing to accept an alternative form of glaucoma treatment through 3-monthly subconjunctival injections (Song et al; 2013. J Glaucoma. 22:190-4).

Subconjunctival injection in humans is a safe technique commonly used for various indications: injection of mitomycin C to decrease intraocular pressure (Gandolfi et al; 1995. Arch Ophthalmol. 113:582-5), corticosteroid injection for the treatment of anterior scleritis (Sen et al; 2005. Br J Ophthalmol. 89: 917-8) or macular oedema (Carbonnibre et al; 2017. J Fr Ophtalmol. 40:177-86). The conjunctiva covers the anterior sclera, the bulbar conjunctiva, and lines the inner side of the eyelids, the palpebral conjunctiva. The conjunctiva is a thin, transparent membrane composed of an epithelium and stroma layers. The average total thickness of the human bulbar conjunctiva is around 240 μm, with an epithelium thickness of =50 μm and a stroma thickness of =190 μm (Zhang et al; 2011. Invest Ophthalmol Vis Sci. 52:7787-91). The subconjunctival injections occurred between the bulbar conjunctiva and the sclera. The injected volumes are usually between 0.1 and 0.5 mL (Subrizi et al; 2019, Drug Discovery Today, 24:1446-57).

Different compositions were investigated for sustained drug delivery after single subconjunctival injection. Their delivery performances were evaluated in vitro and in vivo in rabbit or monkey. Several DDS were prepared with timolol maleate (a beta-blocker). Timolol maleate was incorporated in microfilm implants (4×6 mm) with a thickness of 40 μm made of a copolymer of poly(lactide-co-caprolactone). Then, in monkeys after a limited conjunctival dissection, one timolol loaded microfilm was inserted before suture of the conjunctiva. A sustained intraocular pressure (IOP) reduction was observed for 5 months (Ng et al; 2015. Drug Deliv. and Transl. Res. 5:469-79). Encapsulation of timolol maleate was also done in PLGA degradable microspheres (mean diameter of 14 μm). The sustained delivery of timolol occurred for 3 months in vitro, and after subconjuntival injection in ocular normotensive rabbit, a sustained IOP-lowering effect was measured for 3 months (Lavik et al; 2016. J Ocul Pharmacol Ther. 32:642-49). Huang et al. (J Ocul Pharmacol Ther. 21:445-53; 2005) prepared timolol maleate discs composed of PLGA which reduce IOP only during one week after implantation onto the cul de sac of hypertensive rabbits. One other B-blocker, the brimonidine tartrate was incorporated in PLGA microspheres (average diameter of 7.4 μm). After a single subconjunctival injection in rabbit (150 μL of MS suspension), an ocular hypotensive effect was measured during one month (Fedorchak et al; 2014. Exp Eye Res. 2014 Aug;125:210-6).

Brinzolamide, a carbonic anhydrase inhibitor which decreases the secretion of aqueous humor, was encapsulated in nanoparticles of PLGA. Their subconjunctival injection in normotensive rabbits triggers a reduction for 10 days (Salama et al; 2017. AAPS PharmSciTech. 18:2517-28).

Injectable DDS for sustained delivery of prostaglandin analogues for subconjunctival injections were described. Giarmoukakis et al (Exp Eye Res. 112:29-36; 2013) reported preparation of PLA-PEG nanoparticles (80 nm size) containing latanoprost. In vitro, the release was achieved after one week, and in vivo after minimally invasive subconjunctival injection, a significant hypotensive effect for up to 8 days was obtained in normotensive rabbit. Latanoprost was also encapsulated in liposomes of 100 nm. In vitro, the drug release was sustained for 1 month, and after a single subconjunctival injection in non-human primate, a sustained IOP-lowering effect was observed for 4 months (Natarajan et al; 2014. ACS Nano. 8:419-29).

Preclinical studies demonstrate that a single subconjunctival implantation of different DDS such degradable microfilms, microspheres, liposomes or nanoparticles are efficient to reduce the intra-ocular pressure for several weeks or months. This route of administration of hypotensive drug incorporated in DSS seems efficient. Some of the DDS described above are inappropriate for clinical use, such the solid implants of PLGA which require an incision of conjunctiva for their implantation. The others DDS (liposomes, nanospheres, microspheres) can be implanted min-invasively by injection. Most of them have a size lower than the diameter of the blood vessel present in the bulbar conjunctiva (between 16 and 22 μm) (Shu et al. 2019. Eye and Vision. 6:15). During subconjunctival injection a risk of accidental embolization of retinal and choroidal vessels cannot be excluded. Case of central retinal artery occlusion are described after peribulbar injections of steroids which contain crystals between 1 μm to 1000 μm (Li et al; 2018. Medicine 97:17; Benzon et al; 2007. Anesthesiology. 106:331-8). The mechanism is based on inattentive intra-arterial injection of corticoids and due to the diffuse anastomoses of the facial arterial system, facial injection might cause retrograde embolization with glucocorticoid crystals of ophthalmic or central retinal arteries leading to vision loss. To avoid such accident, after subconjunctival injection, the size and the geometry of DDS containing the anti-glaucoma drugs appears to be key parameters. Ideally, their size should be larger than the diameter of the blood vessels in the conjunctival tissue, but they should be flexible enough to be injected through thin needles.

To locally treat glaucoma the inventors have noticed the interest represented by the local degradable delivery systems.

### SUMMARY OF THE INVENTION

In a first aspect, the invention relates to a composition comprising an effective amount of a prostaglandin analogue, at least one hydrophilic degradable microsphere comprising a crosslinked matrix, and a pharmaceutically acceptable carrier for administration by injection, the crosslinked matrix being based on at least:
a) from 10 to 90 mol % of hydrophilic monomer of general formula (I):

(CH₂═CR₁)—CO-D (I)

wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;

b) from 0.1 to 30 mol % of a cyclic monomer of formula (II):

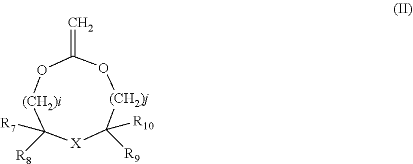

wherein:
R₇, R₈, R₉ and R₁₀ are, independently of one another, hydrogen atom, a (C₁-C₆)alkyl group or an aryl group;
i and j are independently of one another an integer chosen between 0 and 2; and
X is a single bond or an oxygen atom;
   and

c) from 5% to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:

(CH₂═CR₁₁)—CO—Xₙ-PEGₚ-Xₖ—CO—(CR₁₁═CH₂) (IIIa);

W(PEG_{P}-Xₙ—O—CO—(CR₁₁═CH₂))₂ (IIIc);

wherein:
each R₁₁ is independently of one another hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z represents the number of arms of the PEG molecule and is an integer from 3 to 8;

wherein mol % of components a) to c) are expressed relative to the total number of moles of compounds a), b) and c).

In a second aspect, the invention relates to the composition of the invention, for use for preventing and/or treating ocular hypertension or glaucoma.

In a third aspect, the invention relates to the hydrophilic degradable microsphere of the invention for use for delivering an effective amount of a prostaglandin analogue, advantageously of travoprost, to a subject in need thereof.

In a fourth aspect, the invention relates to a pharmaceutical kit comprising:
i) at least one hydrophilic degradable microsphere of the invention in association with a pharmaceutically acceptable carrier for administration by injection;
ii) an effective amount of a prostaglandin analogue, advantageously of travoprost; and
iii) optionally an injection device, the hydrophilic degradable microsphere and the travoprost being packed separately.

### FIGURES

FIG. 1: Effect of microsphere composition on travoprost loading in water for 1 h at room temperature. Comparisons were done relative to the microspheres at 5% crosslinker (PEG₁₃-PLA₇-PCL₃ or PEG₁₃-PCL₈) for the loading objectives at 1 mg/mL (*) or 2 mg/mL (#) using the non-parametric Mann-Whitney test. Significance was set at p<0.05. Data are means.

FIG. 2: Effect of MS composition on travoprost release for the two drug payloads during the swelling of dry microspheres in saline during 10 minutes at room temperature. After freeze-drying, MS were incubated during 10 minutes at room temperature in saline for the two drug payloads. Comparisons between the groups were done using the non-parametric Mann-Whitney test. Significance of the tests was set at p<0.05. NS: non-significant.

FIG. 3: Elution of travoprost during the swelling in saline of the loaded microspheres at 1 mg/mL or 2 mg/mL. A: comparisons were done relative to the microspheres at 5% crosslinker (PEG₁₃-PLA₇-PCL₃ or PEG₁₃-PCL₈) for the 1 mg/mL payload using the non-parametric Mann-Whitney (MW) test (#). The effect of crosslinker composition at 5 mol % or 30 mol % on the drug release was analysed with the non-parametric Mann-Whitney test (*). B: effect of crosslinker composition at 30 mol % on travoprost elution for the 2 mg/mL payload analysed with the non-parametric Mann-Whitney test. Comparisons were done relative to the microspheres at 30% of crosslinker PEG₁₃-PLA₇-PCL₃ (MS2). For each payload, the Kruskal-Wallis (KW) non-parametric test compared the effect of PEG₁₃-PCL₈ crosslinker content on travoprost release. Significance of the tests was set at p<0.05. NS: non-significant. Data are means.

FIG. 4: Elution of travoprost after 2 hours of incubation in PBS of the loaded microspheres at 1 mg/mL or 2 mg/mL. Comparisons were done relative to the microspheres at 30 mol % of crosslinker PEG₁₃-PLA₇-PCL₃ (MS2) using the non-parametric Mann-Whitney test (*). Significance of the tests was set at p<0.05. NS: non-significant. Data are means.

FIG. 5: Effect of crosslinker concentration on the in vitro travoprost release from degradable microspheres at 1 mg/mL.

FIG. 6: Effect of crosslinker concentration and composition on the in vitro travoprost release from degradable microspheres at 2 mg/mL.

FIG. 7: In vitro release during 60 days of travoprost from MS5 after extemporaneous loading (5 min at room temperature).

FIG. 8: Prostaglandin analogues used for the extemporaneous loading on MS5.

FIG. 9: In vitro release in PBS during 5 weeks of latanoprost and latanoprostene BUNOD after the extemporaneous loading on sterile MS5.

FIG. 10: Size distribution of degradable microspheres MS1 (A) and MS3 (B).

### DETAILED DESCRIPTION OF THE INVENTION

The inventors have surprisingly found strong interactions between prostaglandin analogues such as travoprost with hydrophilic degradable microspheres, in particular of size ranging from 50-100 μm, composed of a crosslinked hydrogel.

Prostaglandin analogues are a class of drugs that bind to a prostaglandin receptor. Prostaglandin analogues are used for the treatment of most forms of glaucoma. The compounds should be used whenever low target pressures are called for in both normal-tension glaucoma or primary open-angle glaucoma (POAG), as well as ocular hypertensive (OHT) patients where treatment seems mandatory. The most commonly known prostaglandin analogues are travoprost, latanoprost, bimatoprost and tafluprost.

Travoprost, a prostaglandin analogue, is used to treat open angle glaucoma when other agents are not sufficient. Travoprost is a synthetic analogue of prostaglandin F2a that works by increasing the outflow of aqueous fluid from the eyes. Travoprost concentration of eye drops solutions is 40 μg/mL, and according to a dosage of 1 drop (=50 μL) in the conjunctival sac of the eye once a day, approximatively 2 μg of travoprost are applied to the cornea every day. A local DDS of travoprost must be able to release this amount every day for several months.

The present invention offers the possibility to tune the amount of loaded prostaglandin analogues such as travoprost (25-50-fold higher than commercial topical travoprost) and the flow rate of prostaglandin analogues such as travoprost releases during the time. In addition, the relatively large diameter of the microsphere compared to the anterior art would minimize the retrograde occlusion danger of the retina and of the choroidal vessels during the subconjunctival injection.

The inventors have discovered hydrophilic degradable microspheres that may be used as biocompatible drug carrier for peri-ocular drug delivery and that present affinity with the active ingredient prostaglandin analogues such as travoprost, latanoprost, bimatoprost, latanoprostene BUNOD and tafluprost, in particular travoprost.

The inventors have thus discovered a prostaglandin analogues delivery system that is free of organic solvent, that presents a tuneable degradation time from day to months, that is easy to load (in water, at room temperature), that allows a long-term drug release and that avoids intense inflammatory reaction.

### Definition

As used herein, the expression “matrix based on” means a matrix comprising a mixture of at least components (a) to (c) and/or a matrix resulting from the reaction, in particular from the polymerization, between at least components (a) to (c). Hence, components (a) to (c) can be seen as the starting components that are used for the polymerization (e.g. heterogenous medium polymerization) of the matrix.

The expression “reaction mixture” as used herein designates the polymerisation medium including any components taking part to the polymerisation. The reaction mixture typically comprises at least components a), b), c) as defined in the claims and in this description, optionally a polymerization initiator such as, for example, t-butyl peroxide, benzoyl peroxide, azobiscyanovaleric acid (also called 4,4′-azobis(4-cyanopentanoic acid)), AIBN (azobisisobutyronitrile), or 1,1′-azobis(cyclohexane carbonitrile) or optionally one or more photo-initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5), and at least one solvent, preferably a solvent mixture comprising an aqueous solvent and an organic solvent such as an apolar aprotic solvent, for example a water/toluene mixture and optionally any suitable components as described herein (e.g. stabilizer such as polyvinyl alcohol).

Thus, in the present description, expressions such as “the [starting component X] is added to the reaction mixture in an amount of between YY % and YYYY %” and “the cross-linked matrix is based on [starting component X] in an amount of between YY % and YYYY %” are interpreted in a similar manner. Similarly, expressions such as “the reaction mixture comprises at least [starting component X]” and “the cross-linked matrix is based on at least [starting component X]” are interpreted in a similar manner.

In the context of the invention, “organic phase” of the reaction mixture means the phase comprising the organic solvent and the compounds soluble in said organic solvent, in particular the monomers, and the polymerization initiator.

As used herein, the terms “(Cₓ-C_{y})alkyl group” mean a saturated monovalent hydrocarbon chain, linear or branched, containing X to Y carbon atoms, X and Y being integers between 1 and 36, preferably between 1 and 18, in particular between 1 and 6. Examples are methyl, ethyl, propyl, iso-propyl, butyl, iso-butyl, sec-butyl, tert-butyl, pentyl or hexyl groups.

As used herein, the terms “aryl group” and “(Cₓ-C_{y})aryl” mean an aromatic hydrocarbon group, preferably having X to Y carbon atoms, X and Y being integers between 6 and 36, preferably between 6 and 18, in particular between 6 and 10. The aryl group may be monocyclic or polycyclic (fused rings).Examples are phenyl or naphthyl groups.

As used herein, the terms “partition coefficient P” mean the ratio of concentrations of a compound in a mixture of two immiscible solvents at equilibrium: water and 1-octanol. This ratio is therefore a comparison of the solubilities of the solute in these two liquids. Hence the octanol/water partition coefficient measures how hydrophilic (octanol/water ratio<1) or hydrophobic (octanol/water>1) a compound is. Partition coefficient P may be determined by measuring the solubilities of the compound in water and in 1-octanol and by calculating the ratio solubility in octanol/solubility in water. Partition P may also be determined in silico using Chemicalize provided by ChemAxon.

As used herein, the hydrophobic/hydrophilic balance R of the degradable crosslinkers is quantified by the ratio of the number of hydrophobic units to the number of hydrophilic units according the following equation:

$$R = \frac{{N}_{hydrophobic units}}{{N}_{hydrophilic units}}$$

with N being an integer and representing the number of unit(s).

For example, for the crosslinkers that can be used in the present invention, R is:

$$R = \frac{{N}_{CHlactide} + {N}_{CH 3 lactide} + {N}_{CH 2 glycolide} + 5 \times {N}_{C H 2 c a p rolactone}}{{N}_{EO unit}}$$

with N being an integer and representing the number of unit(s).

As used herein, the terms “degradable microsphere” mean that the microsphere is degraded or cleaved by hydrolysis in a mixture of degradation products composed of low-molecular-weight compounds and water-soluble polymer chains having molecular weights below the threshold for renal filtration of 50 kg mol⁻¹.

As used herein, the expression “hydrophilic degradable microsphere” means a degradable microsphere containing from 10% to 90% of a hydrophilic monomer which allows a good compatibility with the aqueous media and a low adhesion to solid surface (syringes, needles, catheters).

As used herein, the expression “between X and Y” (wherein X and Y are numerical value) means a range of numerical values in which the limits X and Y are inclusive.

As used herein, the expression “immediate release (IR)” of an active ingredient means the rapid release of the active ingredient from the formulation to the location of delivery as soon as the formulation is administered.

As used herein, the expression “extended-release” of an active ingredient means either the “sustained-release (SR)” or the “controlled-release (CR)” of active ingredients from the formulation to the location of delivery at a predetermined rate for an extended period of time and maintaining a constant active ingredient level for this period of time with minimum side effects. The controlled-release (CR) differs from the sustained-release (SR) in that CR maintains drug release over a sustained period at a constant rate whereas SR maintains drug release over a sustained period but not at a constant rate.

As used herein, the expression “sustained-release” of an active ingredient means an extended-release (as defined above) of an active ingredient from the formulation to the location of delivery in order to maintain for a certain predetermined time the drug in tissue of interest at therapeutic concentrations by means of an initial dose portion.

As used herein, the expression “controlled-release (CR)” of an active ingredient means an extended-release (as defined above) of an active ingredient from the formulation to the location of delivery that provides some control of temporal or spatial nature, or both.

As used herein, the term “pharmaceutically acceptable” is intended to mean what is useful to the preparation of a pharmaceutical composition, and what is generally safe and non toxic, for a pharmaceutical use.

As used herein, the terms pharmaceutically acceptable salt mean a salt of a compound which is pharmaceutically acceptable, as defined above, and which possesses the pharmacological activity of the corresponding compound. Such salts comprise:
(1) hydrates and solvates,
(2) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(3) salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Molar Percentage is Abbreviated Herein as Mol %.

Microsphere

According to the present invention, the hydrophilic degradable microsphere comprises a crosslinked matrix that is based on, preferably that results from the polymerization of, at least the following components:
a) from 10 to 90 mol % of a hydrophilic monomer of general formula (I):

(CH₂═CR₁)—CO-D (1)

wherein:
D is O—Z or NH—Z, with Z being —(CR₂R₃)ₘ—CH₃, —(CH₂—CH₂—O)ₘ—H, —(CH₂—CH₂—O)ₘ—CH₃, —(CR₂R₃)ₘ—OH or —(CH₂)ₘ—NR₅R₆ with m being an integer from 1 to 30;
   R₁, R₂, R₃, R₄, R₅ and R₆ are, independently of one another, hydrogen atom or a (C₁-C₆)alkyl group;
b) from 0.1 to 30 mol % of a cyclic monomer of formula (II):

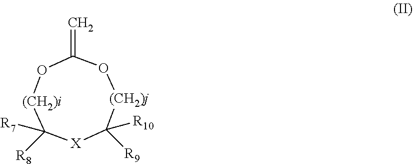

wherein:
R₇, R₈, R₉ and R₁₀ are, independently of one another, hydrogen atom, a (C₁-C₆)alkyl group or an aryl group;
i and j are independently of one another an integer chosen between 0 and 2; and
X is a single bond or an oxygen atom;
   and
c) from 5 mol % to 90 mol % of one degradable block copolymer cross-linker, wherein the degradable block copolymer crosslinker is linear or star-shaped and presents (CH₂═(CR₁₁))—groups at all its extremities, each R₁₁ being independently of one another hydrogen atom or a (C₁-C₆)alkyl group, and wherein the degradable block copolymer crosslinker has a partition coefficient P of between 0.50 and 11.20, or a hydrophobic/hydrophilic balance R between 1 and 20;
   wherein mol % of components a) to c) are expressed relative to the total number of moles of compounds a), b) and c).

The partition coefficient P is determined in silico using Chemicalize provided by ChemAxon.

When the hydrophilic degradable microsphere comprises a crosslinked matrix that is based on further monomers (see monomer e) below), the mol % of components a) to c) are expressed relative to the total number of moles of compounds a), b), c) and e).

The terms “hydrophilic monomer” mean a monomer having a high affinity for water, i.e. tending to dissolve in water, to mix with water, to be wetted by water, or that gives rises to a polymer capable of swelling in water after polymerization.

The block copolymer cross-linker is a degradable block copolymer cross-linker, i.e. a polymer with linear (or radial) arrangement of different blocks joined by covalent bond. In a degradable block copolymer the covalent bond are degradable such as ester bonds, amide bonds, anhydride bonds, urea bonds or polysaccharidic bonds and here specifically ester bonds.

When X is a single bond, it is meant that the carbon atoms bearing R₇, R₈, R₉ and R₁₀ groups are directly linked via a single bond.

The hydrophilic degradable microsphere is a swellable degradable (i.e. hydrolyzable) cross-linked polymer in the form of spherical particle having a diameter after swelling in physiological saline solution (i.e. normal saline solution) ranging between 20 μm and 1200 μm. In particular the polymer of the invention is constituted of at least one chain of polymerized monomers a), b) and c) as defined above.

In the context of the invention, a polymer is swellable if it has the capacity to absorb liquids, in particular water. The expression “size after swelling” means thus that the size of the microspheres is considered after the polymerization and sterilization steps that take place during their preparation.

Advantageously, the microsphere of the invention has a diameter after swelling in physiological saline solution (i.e. normal saline solution) of between 20 μm and 100 μm, 40 μm and 150 μm, 100 μm and 300 μm, 300 μm and 500 μm, 500 μm and 700 μm, 700 μm and 900 μm or 900 μm and 1200 μm, advantageously of between 20 μm and 100 μm, 40 μm and 150 μm, 100 μm and 300 μm, 300 μm and 500 μm, 500 μm and 700 μm as determined by optical microscopy. Microspheres are advantageously small enough in diameter to be injected through needles, catheters or microcatheters with internal diameters ranging from a few hundred micrometres to more than one millimetres.

The hydrophilic monomer a) is of general formula (I):

(CH₂═CR₁)—CO-D (I)

wherein:
D is O—Z or NH—Z, with Z being —(CR₂R₃)ₘ—CH₃, —(CH₂—CH₂—O)ₘ—H, (CH₂—CH₂—O)ₘ—CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group.

Advantageously, the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate; advantageously acrylate terminated poly(ethylene glycol), methacrylate terminated poly(ethylene glycol), methoxy poly(ethylene glycol) methacrylate, butoxy poly(ethylene glycol) methacrylate.

In some embodiments, in the formula (I), when Z is —(CR₂R₃)ₘ—CH₃ or —(CR₂R₃)ₘ—OH, m is preferably an integer from 1 to 6.

In some embodiments, in the formula (I), when Z is —(CR₂R₃)ₘ—CH₃, Z is preferably a C₁-C₆-alkyl group.

In some embodiments, in the formula (I), when Z is —(CR₂R₃)ₘ—OH, R₂ and R₃ are preferably hydrogen and m is an integer from 1 to 6.

In some embodiments, the hydrophilic monomer a) is of general formula (I):

(CH₂═CR₁)—CO-D (1)

wherein:
D is O—Z, with Z being —(CH₂—CH₂—O)ₘ—H or —(CH₂—CH₂—O)ₘ—CH₃, with m being an integer from 1 to 30;
R₁ is hydrogen atom or a (C₁-C₆)alkyl group, preferably a methyl.

More advantageously, the hydrophilic monomer a) is poly(ethylene glycol) methyl ether methacrylate (m-PEGMA).

The amount of hydrophilic monomer a) typically ranges from 10 mol % to 90 mol %, preferably from 30 mol % to 85 mol %, more preferably from 30 mol % to 80 mol %, relative to the total number of moles of components a), b) and c) (or relative to the total number of moles of components a), b), c) and e) when e) is present—see below).

The hydrophilic monomer a) is advantageously present in the reaction mixture in an amount ranging from 10 mol % to 90 mol %, preferably from 30 mol % to 85 mol %, more preferably from 30 mol % to 80 mol %, relative to the total number of moles of components a), b) and c).

Component b) is a cyclic monomer of formula (II) as defined above, wherein:
R₇, R₈, R₉ and R₁₀ are, independently of one another, hydrogen atom, a (C₁-C₆)alkyl group or an aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 2;
X is a single bond or an oxygen atom.

Advantageously, component b) is a cyclic monomer of formula (II) as defined above, wherein:
R₇, R₈, R₉ and R₁₀ are, independently of one another, hydrogen atom or a (C₅-C₇)aryl group;
i and j are independently of one another an integer chosen between 0 and 1;
X is a single bond or an oxygen atom.

Advantageously, the component b) is selected from the group consisting of 2-methylene-1,3-dioxolane, 2-methylene-1,3-dioxane, 2-methylene-1,3-dioxepane, 2-Methylene-1,3,6-Trioxocane and derivatives thereof, in particular benzo derivatives and phenyl substituted derivatives, advantageously from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3-dioxepane, more advantageously from the group consisting of 2-methylene-1,3-dioxepane, 5,6-benzo-2-methylene-1,3-dioxepane and 2-methylene-1,3,6-trioxocane. More advantageously, the component b) is 2-methylene-1,3-dioxepane or 2-methylene-1,3,6-trioxocane.

The amount of component b) typically ranges from 0.1 mol % to 30 mol %, preferably from 1 mol % to 20 mol %, and in particular from 1 mol % to 10 mol %, relative to the total number of moles of components a), b) and c) (or relative to the total number of moles of components a), b), c) and e) when e) is present—see below). In some embodiments, the amount of component b) is about 10 mol %.

The cyclic monomer b) of general formula (II) is advantageously present in the reaction mixture in an amount ranging from 0.1 mol % to 30 mol %, preferably from 1 mol % to 20 mol %, and in particular from 5 mol % to 15 mol % or from 1 mol % to 10 mol %, relative to the total number of moles of components a), b) and c). In some embodiments, the amount of component b) is about 10 mol %.

Component c) is a degradable block copolymer crosslinker, wherein the degradable block copolymer crosslinker is linear or star-shaped and presents (CH₂═(CR₁₁))-groups at all its extremities, each R₁₁ being independently of one another hydrogen atom or a (C₁-C₆)alkyl group.

The degradable block copolymer crosslinker has a partition coefficient P of between 0.50 and 11.20, advantageously between 3.00 and 9.00. Or the degradable block copolymer crosslinker has a hydrophobic/hydrophilic balance R between 1 and 20, advantageously between 3 and 15.

As used herein, the expression “copolymer cross-linker” is intended to mean that the copolymer contains a functional group containing a double bond at least two of its extremities in order to link together several polymer chains.

The cross-linker c) as defined above is linear or star-shaped (advantageously from 3 to 8 arms) and it presents (CH₂═(CR₁₁))-groups at all its extremities (i.e. at its two extremities when linear and at the end of each arm when star-shaped), each R₁₁ being independently of one another hydrogen atom or a (C₁-C₆)alkyl group, preferably a methyl group. Advantageously, the crosslinker c) presents (CH₂═(CR₁₁))—CO—at all its extremities, each R₁₁ being independently of one another hydrogen atom or a (C₁-C₆)alkyl group, preferably a methyl group. Advantageously, all the R₁₁ are identical and are hydrogen atom or a (C₁-C₆)alkyl group, preferably a methyl group.

The crosslinker c) is of general formula (IIIa) or (IIIc) as follows:

(CH₂═CR₁₁)—CO—Xₙ—PEG_{P}-Xₖ—CO—(CR₁₁═CH₂) (IIIa);

W(PEG_{P}-Xₙ—O—CO—(CR₁₁═CH₂))₂ (IIIc);

wherein:
each R₁₁ is independently of one another hydrogen atom or a (C₁-C₆)alkyl group;
X independently represents PLA, PGA, PLGA, PCL or PLAPCL;
n, k and p respectively represent the degree of polymerization of X, and PEG, n and k independently being integers from 1 to 150, and p being an integer from 1 to 100;
W is a carbon atom, a C₁-C₆-alkyl group (preferably a C₁-C₃-alkyl) or an ether group comprising 1 to 6 carbon atoms, preferably 1 to 3 carbon atoms;
z is an integer from 3 to 8.

Crosslinker c) of formula (IIIc) is a star-shaped polymer, i.e., a polymer consisting of several linear chains (also designated arms) connected a central core. In the crosslinker of formula (IIIc), W is the core of the star-shaped polymer and —(PEG_{P}Xₙ—O—CO—(CR₁₁═CH₂)) is an arm of the star-shaped polymer with z being the number of arms.

Advantageously, when the crosslinker c) is of general formula (IIIc), n may be identical or different in each arm of the PEG.

In the context of the invention, the abbreviations used herein have the following meaning:

| Abb. | Name | | Formula |
|---|---|---|---|
| PEG | polyethylene glycol | PEGₚ | 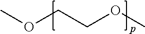 |
| PLA | poly-lactic acid (also named poly-lactide) | PLA_{n or k} | 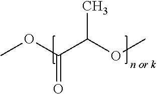 |
| PGA | poly-glycolic acid (also named poly-glycolide) | PGA_{n or k} | 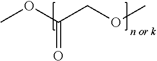 |
| PLGA | poly-lactic-glycolic acid The copolymer comprises both lactide and glycolide units, the degree of polymerization is the sum of the number of lactide and glycolide units | PLGA_{n or k} | 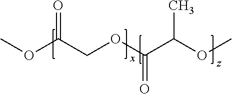 |
| PCL | poly(caprolactone) | PCL_{n or k} | 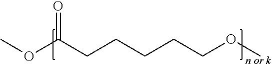 |
| PLAPCL | poly-lactic acid poly- caprolactone The copolymer comprises both lactide and caprolactone units, the degree of polymerization is the sum of the number of lactide and caprolactone units | PLAPCL_{n or k} | 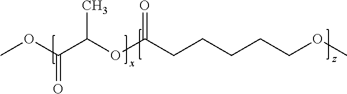 |
| | | | X + Z = n or X + Z = k |

In the above table, n, p and k have the values disclosed herein.

Advantageously, the crosslinker c) is of general formula (IIIa) or (IIIc), in particular (IIIa), as defined above, wherein X represents PLAPCL or PCL. More advantageously, the crosslinker c) is of general formula (IIIa) or (IIIc), in particular (IIIa), wherein X represents PCL.

Advantageously, the crosslinker c) is of general formula (IIIa) or (IIIc), in particular (IIIa), as defined above, wherein n and k independently are integers from 1 to 150, preferably from 1 to 20, more preferably from 1 to 10, even more preferably from 4 to 7. Preferably n+k ranges from 5 to 15 or from 8 to 14 and p is an integer from 1 to 100, preferably from 1 to 20.

Advantageously, the crosslinker c) is of general formula (IIIa) or (IIIc), in particular (IIIa) as defined above, wherein the R₁₁ are identical and are H or a (C₁-C₆)alkyl group.

Advantageously, the crosslinker c) is selected from the group consisting of compounds of general formula (IIIa) or (IIIc), in particular (IIIa), as defined above, wherein:
X=PLA, n+k=12 and p=13 (such as PEG₁₃-PLA₁₂ wherein R₁₁ is methyl);
X=PLAPCL, n+k=10 and p=13 (such as PEG₁₃-PLA₈-PCL₂ or PEG₁₃-PLA₇-PCL₃, wherein R₁₁ is methyl);
X=PLAPCL, n+k=9 and p=13 (such as PEG₁₃-PLA₄-PCL₅, wherein R₁₁ is methyl);
X=PLAPCL, n+k=8 and p=13 (such as PEG₁₃-PLA₂-PCL₆, wherein R₁₁ is methyl);
X=PCL; n+k=8 and p=13 (such as PEG₁₃-PCL₈, wherein R₁₁ is methyl);
X=PLGA; n+k=12 and p=13 (such as PEG₁₃-PLGA₁₂, wherein R₁₁ is methyl);
X=PCL, n+k=10 and p=4 (such as PEG₄-PCL₁₀, wherein R₁₁ is methyl); or
X=PCL, n+k=12 and p=2 (such as PEG₂-PCL₁₂, wherein R₁₁ is methyl).

In these embodiments, R₁₁ is preferably hydrogen or methyl.

In some embodiments, the crosslinker c) is a compound of general formula (IIIc), as defined above, wherein p is 7, X=PLAPCL, n=10, z is 3 with R₁₁ being preferably hydrogen or methyl (such as PEG 3-arm-PLA₇-PCL₃, wherein R₁₁ is methyl).

In some embodiments, the crosslinker c) is selected from the group consisting of compounds of general formula (IIIa), as defined above, wherein:
X=PLAPCL, n+k=10 and p=13 (such as PEG₁₃-PLA₇-PCL₃, wherein R₁₁ is methyl);
X=PCL; n+k=8 and p=13 (such as PEG₁₃-PCL₈, wherein R₁₁ is methyl);
X=PCL, n+k=12 and p=2 (such as PEG₂-PCL₁₂, wherein R₁₁ is methyl).

In these embodiments, R₁₁ is preferably hydrogen or methyl.

Within the definitions of the crosslinker c) above, the polyethylene glycol (PEG) has a number average molecular weight (Mn) of 100 to 10 000 g/mol, preferably 100 to 2 000 g/mol, more preferably 100 to 1 000 g/mol.

The amount of crosslinker c) typically ranges from 5 mol % to 90 mol %, preferably from 5 mol % to 60 mol %, more preferably from 15 mol % to 60 mol %, relative to the total number of moles of components a), b) and c) (or relative to the total number of moles of components a), b), c) and e) when e) is present—see below).

The crosslinker c) is advantageously present in the reaction mixture in an amount ranging from 5 mol % to 90 mol %, preferably from 5 mol % to 60 mol %, more preferably from 15 mol % to 60 mol % relative to the total number of moles of components a), b) and c).

Increasing the amount of crosslinker, and thus decreasing the mesh size of the resulting microsphere, influences the loading of the microsphere in prostaglandin analogues, such as travoprost, and then the release of the prostaglandin analogues, such as travoprost. For amount greater than 15 mol %, an optimal release of the travoprost is achieved, in particular because it prevents the immediate release of a large part of the travoprost.

The crosslinked matrix of the hydrophilic degradable microsphere is advantageously further based on a chain transfer agent d), preferably results from the polymerization of components a), b) and c) in presence of a chain transfer agent d).

For the purposes of this invention, “transfer agent” means a chemical compound having at least one weak chemical bond. This agent reacts with the radical site of a growing polymer chain and interrupts the growth of the chain. In the chain transfer process, the radical is temporarily transferred to the transfer agent which restarts growth by transferring the radical to another polymer or monomer.

Advantageously, the chain transfer agent d) is selected from the group consisting of monofunctional or polyfunctional thiols, alkyl halides, transition metal salts or complexes and other compounds known to be active in free radical chain transfer processes such as 2,4-diphenyl-4-methyl-1-pentene. More advantageously, the chain transfer agent is a cycloaliphatic or aliphatic, thiol preferably having from 2 to 24 carbon atoms, more preferably between 2 and 12 carbon atoms, and having or not a further functional group selected from the groups amino, hydroxy and carboxy.

Advantageously, the chain transfer agent d) is selected from the group consisting of thioglycolic acid, 2-mercaptoethanol, dodecane thiol and hexane thiol.

The amount of chain transfer agent d) typically ranges from 0.1 to 10 mol %, preferably from 2 to 5 mol %, relative to the number of moles of monomer a).

The chain transfer agent d) is advantageously present in the reaction mixture in an amount of, for example, from 0.1 to 10 mol %, preferably from 2 to 5 mol %, relative to the number of moles of monomer a).

In a particular aspect of the invention, the crosslinked matrix is only based on starting components a), b), c) and optionally d), as defined above and in the contents abovementioned, no other starting component are thus added to the reaction medium. It is thus clear that the sum of the above-mentioned contents of monomers (components (a), (b) and (c)) must be equal to 100%.

In some embodiments, the crosslinked matrix is advantageously further based on at least one ionised or ionisable monomer e) of general formula (V):

(CH₂═CR₁₂)-M-E (V),

wherein:
R₁₂ is hydrogen atom or a (C₁-C₆)alkyl group;
M is a single bond or a divalent radical having 1 to 20 carbon atoms, advantageously a single bond;
E is a ionised or ionisable group being advantageously selected from the group consisting of —COOH, —COO⁻, —SO₃H, —SO₃⁻, —PO₄H₂, —PO₄H⁻, —PO₄²⁻, —NR₁₃R₁₄, and —NR₁₅R₁₆R₁₇⁺; R₁₃, R₁₄, R₁₅, R₁₆ et R₁₇ being independently of one another hydrogen atom or a (C₁-C₆)alkyl group.

In the context of the invention, an ionised or ionisable group is understood to be a group which is charged or which may be in charged form (in the form of an ion), i.e. which carries at least one positive or negative charge, depending on the pH of the medium. For example, the COOH group may be ionised in the COO⁻ form, and the NH₂ group may be ionised in the form of NH₃⁺.

The introduction of an ionised or ionisable monomer into the reaction media makes it possible to increase the hydrophilicity of the resulting microspheres, thereby increasing the swelling rate of said microspheres, further facilitating their injection via catheters and microcatheters. In addition, the presence of an ionised or ionisable monomer improves the loading of active substances into the microsphere.

In an advantageous embodiment, the ionised or ionisable monomer e) is a cationic monomer, advantageously selected from the group consisting of 2-(methacryloyloxy)ethyl phosphorylcholine, 2-(dimethylamino)ethyl (meth)acrylate, 2-(diethylamino)ethyl (meth)acrylate and 2-((meth)acryloyloxy)ethyl] trimethylammonium chloride, more advantageously the cationic monomer is diethylamino)ethyl (meth)acrylate. Advantageously, the ionised or ionisable monomer e) is present in the reaction mixture in an amount of between 0% and 30% by mole, advantageously between 1% and 30% by mole, preferably from between 10% and 15% by mole, relative to the total number of moles of the monomers (components a)+b)+c)+e)). It is thus clear that in such a case the sum of the above-mentioned contents of monomers (components (a), (b) and (c) and (e)) must be equal to 100%.

In another advantageous embodiment, the ionised or ionisable monomer e) is an anionic monomer advantageously selected from the group consisting of acrylic acid, methacrylic acid, 2-carboxyethyl acrylate, 2-carboxyethyl acrylate oligomers, 3-sulfopropyl (meth)acrylate potassium salt and 2-(methacryloyloxy)ethyl]dimethyl-(3-sulfopropyl)ammonium hydroxide, more advantageously, the anionic monomer is acrylic acid. The amount of ionised or ionisable monomer e) typically ranges from 0 or from 0.1 to 30 mol %, preferably from 10 to 15% by mole, relative to the total number of moles of the monomers (components a)+b)+c)+e)). It is thus clear that in such a case the sum of the above-mentioned contents of monomers (components (a), (b) and (c) and (e)) must be equal to 100%. Advantageously, the ionised or ionisable monomer e) is present in the reaction mixture in an amount that ranges from 0 mol % to 30 mol %, advantageously from 1 mol % to 30 mol %, preferably from 10 mol % to 15 mol %, relative to the total number of moles of the monomers ((components a)+b)+c)+e)).

Advantageously, the ionised or ionisable monomer e) is acrylic acid and is advantageously present in the reaction mixture in an amount of between 0 and 30% by mole, advantageously between 1 and 30% by mole, preferably from between 10 and 15% by mole, relative to the total number of moles of the monomers.

In some embodiments, the hydrophilic degradable microsphere comprises a crosslinked matrix that is based on at least, preferably that results from the polymerization of, the following components:
a) from 10 to 90 mol % of a hydrophilic monomer of general formula (I):

(CH₂═CR₁)—CO-D (I)

wherein:
D is O—Z, Z being —(CH₂—CH₂—O)ₘ—H or —(CH₂—CH₂—O)ₘ—CH₃, with m being an integer from 1 to 30;
   R₁ is hydrogen atom or a (C₁-C₆)alkyl group, preferably a methyl; preferably m-PEGMA,
b) from 0.1 to 30 mol % of a cyclic monomer of formula (II):

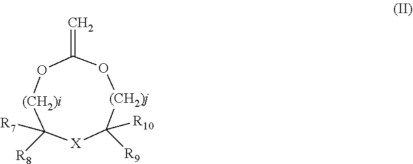

wherein:
R₇, R₈, R₉ and R₁₀ are, independently of one another, hydrogen atom or a (C₅-C₇)aryl group;
   i and j are independently of one another an integer chosen between 0 and 1;
   X is a single bond or an oxygen atom; preferably 2-methylene-1,3-dioxepane;
      and
c) from 5 to 90 mol % of a degradable block copolymer cross-linker of formula:

(CH₂═CR₁₁)—CO—Xₙ—PEGₚ-Xₖ—CO—(CR₁₁═CH₂) (IIIa), or

W(PEG_{P}-Xₙ—O—CO—(CR₁₁═CH₂))_{z} (IIIc);

wherein R₁₁, X, W, n, p, k, z are as disclosed herein,
preferably wherein
R₁₁ is independently of one another hydrogen atom or a (C₁-C₆)alkyl group;
   X=PLA, n+k=12 and p=13 (such as PEG₁₃-PLA₁₂ wherein R₁₁ is methyl); or
   X=PLAPCL, n+k=10 and p=13 (such as PEG₁₃-PLA₈-PCL₂ or PEG₁₃-PLA₇-PCL₃, wherein R₁₁ is methyl); or
   X=PLAPCL, n+k=9 and p=13 (such as PEG₁₃-PLA₄-PCL₅, wherein R₁₁ is methyl); or
   X=PLAPCL, n+k=8 and p=13 (such as PEG₁₃-PLA₂-PCL₆, wherein R₁₁ is methyl); or
   X=PLGA; n+k=12 and p=13 (such as PEG₁₃-PLGA₁₂, wherein R₁₁ is methyl); or
   X=PCL; n+k=8 and p=13 (such as PEG₁₃-PCL₈, wherein R₁₁ is methyl); or
   X=PCL, n+k=10 and p=4 (such as PEG₄-PCL₁₀, wherein R₁₁ is methyl); or
   X=PCL, n+k=12 and p=2 (such as PEG₂-PCL₁₂, wherein R₁₁ is methyl);
and wherein the degradable block copolymer crosslinker has a partition coefficient P between 0.5 and 11.2 or a hydrophobic/hydrophilic balance R between 1 and 20;

wherein mol % of components a) to c) are expressed relative to the total number of moles of compounds a), b) and c).

When the hydrophilic degradable microsphere comprises a crosslinked matrix that is based on further monomers (see monomer e) below), the mol % of components a) to c) are expressed relative to the total number of moles of compounds a), b), c) and e).

The amounts of components a), b) and c) may be as disclosed herein.

The microsphere of the invention can be readily synthesized by numerous methods well-known to the one skilled in the art. By way of example, the microsphere of the invention can be obtained by direct or inverse suspension polymerization as described below and in the Examples or by microfluidic.

A direct suspension may proceed as follows:
(1) stirring or agitating a mixture comprising
   (i) at least the starting components a), b) and c) as defined above;
   (ii) a polymerization initiator present in amounts ranging from 0.1 to approximately 2 parts per weight per 100 parts by weight of the monomers;
   (iii) a surfactant in an amount no greater than about 5 parts by weight per 100 parts by weight of the aqueous solution, preferably no greater than about 3 parts by weight and most preferably in the range of 0.5 to 1.5 parts by weight;
   (iv) a salt in an amount no greater than about 10 parts by weight per 100 parts by weight of the aqueous solution, preferably no greater than about 5 parts by weight and most preferably in the range of 1 to 4 parts by weight; and (v) water to form an oil in water suspension; and
(2) polymerizing the starting components.

In such a direct suspension polymerization, the surfactant may be selected from the group consisting of hydroxyethylcellulose, polyvinyl alcohol (PVA), polyvinylpyrrolidone, polyethylene oxide, polyethylene glycol and polysorbate 20 (Tween® 20).

An inverse suspension may proceed as follows:
(1) stirring or agitating a mixture comprising:
   (i) at least the starting components a), b) and c) as defined above;
   (ii) a polymerization initiator present in amounts ranging from 0.1 to approximately 2 parts per weight per 100 parts by weight of the monomers;
   (iii) a surfactant in an amount no greater than about 5 parts by weight per 100 parts by weight of the oil phase, preferably no greater than about 3 parts by weight and most preferably in the range of 0.5 to 1.5 parts by weight; and
   (iv) oil to form a water in oil suspension;
      and
(2) polymerizing the starting components.

In such a reverse suspension process, the surfactant may be selected from the group consisting of sorbitan esters such as sorbitan monolaurate (Span® 20), sorbitan monopalmitate (Span® 40), sorbitan monooleate (Span® 80), and sorbitan trioleate (Span® 85), hydroxyethyl cellulose, mixture of glyceryl stearate and PEG stearate (Arlacel®) and cellulose acetate.

In the above processes, the polymerization initiator may include t-butyl peroxide, benzoyl peroxide, azobiscyanovaleric acid (also known as 4,4′-azobis(4-cyanopentanoic acid)), AIBN (azobisisobutyronitrile), or 1,1′ azobis (cyclohexane carbonitrile) or one or more photo-initiators such as 2-hydroxy-4′-(2-hydroxyethoxy)-2-methylpropiophenone (106797-53-9); 2-hydroxy-2-methylpropiophenone (Darocur® 1173, 7473-98-5); 2,2-dimethoxy-2-phenylacetophenone (24650-42-8); 2,2-dimethoxy-2-phenyl acetophenone (Irgacure®, 24650-42-8) or 2-methyl-4′-(methylthio)-2-morpholinopropiophenone (Irgacure®, 71868-10-5).

Further, the oil may be selected from paraffin oil, silicone oil and organic solvents such as hexane, cyclohexane, ethyl acetate or butyl acetate.

Travoprost loading may proceed by numerous methods well-known to one of skill in the art such as passive adsorption (swelling of the polymer into a drug solution).

In order to increase the drug loading and control the rate of drug release, a concept consists to introduce certain chemical moieties into the polymer backbone that are capable of interacting with the drug via non covalent interactions. Examples of such interactions include electrostatic interactions (described after), hydrophobic interactions, π-π stacking, and hydrogen bonding, among others.

Drug

The composition comprises an effective amount of a prostaglandin analogue such as travoprost, latanoprost, bimatoprost and tafluprost, in particular travoprost.

Advantageously, the prostaglandin analogue is selected from travoprost, latanoprost, bimatoprost and tafluprost. Advantageously, the prostaglandin analogue is travoprost.

Advantageously, in the composition of the invention, the prostaglandin analogues, in particular travoprost, is loaded/absorbed onto the microsphere as defined above by non-covalent interactions. This particular way of entrapping drugs or prodrugs is called physical entrapment.

Loading of a prostaglandin analogue, in particular travoprost, onto the microsphere of the invention may be proceeded by numerous methods well-known to the one skilled in the art such as preloading a prostaglandin analogue, in particular travoprost, after the microsphere synthesis.

Advantageously, the composition of the invention comprises between 1 and 6 mg/mL of a prostaglandin analogue, in particular travoprost, more advantageously between 2 and 4 mg/mL.

Advantageously, the composition of the invention releases the prostaglandin analogue, in particular the travoprost, without a burst, less 10% during the first day, followed by a constant delivery rate between 1% and 5% of initial loading every day.

Advantageously, after implantation in living organisms, the composition of the invention releases the prostaglandin analogue, in particular the travoprost, in lachrymal fluid without a burst during the first hour following subconjunctival implantation. Concentration of the prostaglandin analogue, in particular travoprost, could remain in the therapeutic range in aqueous humor for 1 to 7 days, advantageously for 1 to 30 days, preferably for 1 to 90 days, the therapeutic range being between 2 ng/mL to 3 ng/mL (Martinez-de-la-Casa et al; 2012. Eye. 26:972-75) or preferably with low plasma concentration, in the same range as observed after topical treatment (<25 μg/mL) with eye-drops.

Composition

The composition comprises an effective amount of a prostaglandin analogue, such as travoprost, latanoprost, bimatoprost and tafluprost, in particular travoprost (0.1-0.6% in mass relative to the microsphere), at least one hydrophilic degradable microsphere as defined above, and a pharmaceutically acceptable carrier. The carrier is suitable for administration by injection.

The prostaglandin analogues, in particular the travoprost, and the hydrophilic degradable microsphere are as defined above.

According to the invention, the pharmaceutically acceptable carrier is intended for administration of a the prostaglandin analogue, in particular the travoprost, by injection and is advantageously selected in the group consisting in water for injection, saline, glucose, starch, hydrogel, polyvinylpyrrolidone, polysaccharide, hyaluronic acid ester, contrast agent and plasma.

The formulations may be administered by subconjunctival injection. The formulations of hydrophilic degradable microspheres are syringable, the microsphere size and distribution are shown in FIG. 5 for example. This enables the administration in a needle that is from between 21 and 34 gauge.

The composition of the invention can also contain a buffering agent, a preservative, a gelling agent, a surfactant, or mixtures thereof. Advantageously, the pharmaceutically acceptable carrier is saline or water for injection.

The composition of the invention allows the sustained release of the prostaglandin analogue, in particular travoprost, over a period ranging from a few hours to a few months. Advantageously, the composition of the invention allows the sustained-release of the prostaglandin analogue, in particular travoprost, for at least 4 weeks without burst, in particular between 4 weeks and 6 months, more particularly between 4 weeks and 3 months.

The composition of the invention allows the control of the sustained-release as defined above, for example by modulating the nature and the contents of monomers a), b) and/or c) and the amount of loaded the prostaglandin analogue, in particular travoprost.

The invention also relates to the composition as defined above, for use for preventing and/or treating ocular hypertension or glaucoma.

The invention also relates to a method for preventing and/or treating ocular hypertension or glaucoma, comprising administering to a subject in need thereof an effective amount of the composition as defined above.

The invention also relates to the use of the composition as defined above for the manufacturing of a drug for preventing and/or treating ocular hypertension or glaucoma.

Extemporaneous Loading

In a particular embodiment of the invention, the travoprost may be loaded extemporaneously on dry and sterile microsphere.

The invention thus also relates to a pharmaceutical kit comprising:
i) at least one hydrophilic degradable microsphere as defined above in association with a pharmaceutically acceptable carrier for administration by injection;
ii) an effective amount of travoprost; and
iii) optionally an injection device, the hydrophilic degradable microsphere and the travoprost being packed separately.

in such an embodiment, the travoprost is advantageously intended to be loaded on the hydrophilic degradable microsphere just before the injection.

According to the present invention, “injection device” means any device for parenteral administration. Advantageously, the injection device is one or more syringes, which may be pre-filled, and/or one or more catheters or microcatheters.

Use of the Microsphere

The invention also relates to the hydrophilic degradable microsphere as defined above for use for the delivery, advantageously the sustained-delivery, of an effective amount of travoprost to a subject in need thereof.

Advantageously, the sustained delivery of travoprost is over a period ranging from a few weeks to a few months without burst, advantageously for at least 4 weeks, in particular between 4 weeks and 6 months, more particularly between 4 weeks and 3 months.

The composition of the invention allows the sustained release of travoprost over a period ranging from a few hours to a few months. Advantageously, the composition of the invention allows the sustained-release of travoprost for at least 4 weeks without burst, in particular between 4 weeks and 6 months, more particularly between 4 weeks and 3 months.

The examples which follow illustrate the invention without limiting its scope in any way.

### EXAMPLES

### Example 1: Preparation of Unloaded Microsphere According to the Invention

The starting components, their contents and the main parameters for microspheres synthesis are summarized in Tables 1a and 1b.

TABLE 1a
Formulations of microspheres according to the invention

| | Microsphere of 50-100 μm diameters | | | | | | | |
|---|---|---|---|---|---|---|---|---|
| | Test number | | MS1 | MS2 | MS3 | MS4 | MS5 | MS6 |
| Process | Oil/Water | | 1/11 | 1/11 | 1/11 | 1/11 | 1/11 | 1/11 |
| Para- | ratio (V/V) | | | | | | | |
| meters | Stirring speed | | 240 | 240 | 240 | 240 | 240 | 240 |
| | | | RPM | RPM | RPM | RPM | RPM | RPM |
| | PVA | | 1% | 1% | 1% | 1% | 1% | 1% |
| | NaCl | | 3% | 3% | 3% | 3% | 3% | 3% |
| Organic | Monomers mass/ | | 56% | 56% | 56% | 56% | 56% | 56% |
| phase | organic phase | | | | | | | |
| | mass (wt %) | | | | | | | |
| | Toluene | | 44% | 44 % | 44% | 44% | 44% | 44% |
| | Hexanethiol | | 3% | 3% | 3% | 3% | 3% | 3% |
| | (component d) | | | | | | | |
| | (% mole/m- | | | | | | | |
| | PEGMA mole) | | | | | | | |
| | AIBN | | | | | | | |
| | (% weight/weight | | | | | | | |
| | organic phase | | 0.28% | 0.28% | 0.28% | 0.28% | 0.28% | 0.28% |
| | Phase | m-PEGMA | 85% | 60% | 85% | 75% | 60% | 40% |
| | | (component a) | | | | | | |
| | | (% mole/total | | | | | | |
| | | mole | | | | | | |
| | | monomer) | | | | | | |
| | | Crosslinker | 5% of | 30% | 5% of | 15% | 30% | 50% |
| | | (component c) | PEG₁₃- | of | PEG₁₃- | of | of | of |
| | | (% mole/total | PLA₇- | PEG₁₃- | PCL₈ | PEG₁₃- | PEG₁₃- | PEG₁₃- |
| | | mole | PCl₃ | PLA₇- | | PCL₈ | PCL₈ | PCL₈ |
| | | monomer) | | PCL₃ | | | | |
| | | 2-methylene- | 10% | 10% | 10% | 10% | 10% | 10% |
| | | 1,3-dioxepane | | | | | | |
| | | (MDO) | | | | | | |
| | | (component b) | | | | | | |
| | | (% mole/total | | | | | | |
| | | mole | | | | | | |
| | | monomer) | | | | | | |

TABLE 1b
Formulations of microspheres according to the invention

| | | | Microspheres of 50-100 μm diameter | | |
|---|---|---|---|---|---|
| | Test number | | MS7 | MS8 | MS9 |
| Process | Oil/Water ratio (V/V) | | 1/11 | 1/11 | 1/11 |
| parameters | Stirring speed | | 240 RPM | 240 RPM | 240 RPM |
| | PVA | | 1% | 1% | 1% |
| | NaCl | | 3% | 3% | 3% |
| Organic | Monomers mass/organic phase | | 56 | 56 | 56 |
| phase | mass (wt %) | | | | |
| | Toluene | | 44 | 44 | 44 |
| | Hexanethiol (component d) | | 3 | 3 | 3 |
| | (% mole/m-PEGMA mole or tert- | | | | |
| | butyl methacrylate) | | | | |
| | AIBN | | 0.28 | 0.28 | 0.28 |
| | (% weight/weight organic phase | | | | |
| | Phase | m-PEGMA | 85% | 0% | 60% |
| | monomer | (component a) | | | |
| | | (% mole/total mole | | | |
| | | monomer) | | | |
| | | Tert-butyl methacrylate | 0% | 60% | 0% |
| | | (component a) | | | |
| | | (% mole/total mole | | | |
| | | monomer) | | | |
| | | Crosslinker | 5% | 30% | 30% |
| | | (component c) | of PEG 3-arm- | of PEG₁₃-PLA₇- | of PEG₂-PCL₁₂ |
| | | (% mole/total mole | PLA₇-PCL₃ * | PCL₃ | |
| | | monomer) | | | |
| | | 2-methylene-1,3- | 10% | 10% | 10% |
| | | dioxepane (MDO) | | | |
| | | (component b) | | | |
| | | (% mole/total mole | | | |
| | | monomer) | | | |

* the crosslinker is 3 arm PEG with a molar mass of 1014 g/mol, PLA₇-PCl₃ as a total of 10 units.

The aqueous phase solution (917 mL) containing 1 wt % polyvinyl alcohol (Mw=13000-23000 g/mol), 3 wt % NaCl in deionized water was placed in a 1 dm³ reactor and heated up to 50° C.

The organic phase was prepared in an Erlenmeyer. Briefly, toluene (36.9 g) and 2,2′-azobis(2-methylpropionitrile) (AIBN) (0.28 wt %/organic phase weight) were weighted. AIBN was introduced in another vial and solubilized in a volume fraction (=30%) of the weighted toluene.

Then, degradable crosslinker was weighted in an Erlenmeyer. Polyethylene glycol methyl ether methacrylate (Mn=300 g/mol) or tert-butyl methacrylate (Mn=142.2 g/mol) and 2-methylene-1,3-dioxepane (MDO) were weighted and introduced into the Erlenmeyer. Then the remaining volume of toluene was added to solubilize the monomers. Hexanethiol (3% mol/mol of m-PEGMA or tert-butyl methacrylate) was added to the Erlenmeyer. The AIBN solution in toluene was added to the Erlenmeyer containing monomers. Finally, the organic phase had to be clear (monomer and initiator should be totally solubilized) without any aggregates before introduction into the aqueous phase.

The organic phase was poured into the aqueous phase at 50° C. Thereupon, stirring (240 rpm) was applied by using an impeller. After 4 minutes, the temperature had raised up to 80° C. After 8 hours, the stirring was stopped and microspheres were collected by filtration on a 40 μm sieve and washed extensively with acetone and water. Microspheres were then sieved with decreasing sizes of sieves (125, 100, 50 μm). MS in the size range 50-100 μm were collected for drug loading trials.

### Example 2: Loading of Microspheres According to Example 1 with Travoprost (Preloading after Ms Synthesis)

After the sieving step, 250 μL of microspheres obtained in example 1 (size range 50-100 μm) were placed in 15 mL polypropylene vials. Then, water (500 μL or 1 mL) was added, before the addition of 500 μL or 1 mL, respectively, of travoprost solution (Sigma, PHR1622-3ML, #LRAA5292 (0.499 μg/mL in water/acetonitrile (70/30)).

The loading step was done at room temperature for 1 h under stirring on a tube rotator (=30 rpm). Then, the supernatants were removed for the measurement of unbound travoprost by fluorimetry (λₑₓ 220 nm, λ_{Em} 310 nm). The amount of travoprost in supernatant was obtained by extrapolation from a standard curve (0.6 to 20 μg/mL), and the loaded dose was calculated by subtraction. The loaded dose was calculated by subtracting the final amount of travoprost from the initial amount. The travoprost loading for 1 mL of beads was obtained by multiplying by 4 the quantity loaded on 0.25 mL of MS. The loading efficiency was calculated by the following equation: loading efficiency=((Travoprost in feed—Travoprost in supernantant)/Travoprost in feed)×100. The pellets were washed with 2 mL of glucose (2.5% in water) before freeze-drying.

Table 2 and FIG. 1 summarizes travoprost loading for each MS formulation tested.

TABLE 2
Travoprost loading on the microspheres of
example 1 according to example 2.

| | Travoprost loading | Travoprost loading | |
|---|---|---|---|
| | (mg/mL) | (mg/mL) | Log P |
| | for 0.5 mL of | for 1 mL of travoprost | value for |
| Test | travoprost solution | solution | degradable |
| number | (% of loading efficacy) | (% of loading efficacy) | crosslinker |
| MS1 | 0.93 ± 0.01 | 1.79 ± 0.004 | 3.2 |
| | (93%) | (90%) | |
| MS2 | 0.98 ± 0.001 | 1.95 ± 0.001 | 3.2 |
| | (99%) | (97%) | |
| MS3 | 0.94 ± 0.004 | 1.86 ± 0.0028 | 6.5 |
| | (95%) | (93%) | |
| MS4 | 0.98 ± 0.0003 | 1.95 ± 0.009 | 6.5 |
| | (99%) | (98%) | |
| MS5 | 0.98 ± 0.003 | 1.96 ± 0.001 | 6.5 |
| | (99%) | (98%) | |
| MS6 | 0.99 ± 0.001 | 1.97 ± 0.003 | 6.5 |
| | (99%) | (98%) | |
| MS7 | ND | 1.87 ± 0.007 | 3.2 |
| | | (94%) | |
| MS8 | 0.99 ± 0.001 | ND | 3.2 |
| | (99%) | | |
| MS9 | ND | 1.96 ± 0.001 | 11.2 |
| | | (98%) | |

The travoprost solution used for the loading experiments was from Sigma (PHR1622-3ML at 0.499 μg/mL in water/acetonitrile mixture (70/30)).
ND: not determined.

The loading of increasing amounts of travoprost was achievable with yields higher than 90% on preformed microspheres synthesized according to example 1. The loading efficiency was significantly improved when the crosslinker content in the microspheres was higher than 5% (Table 2 & FIG. 1).

For the MS containing the degradable crosslinker PEG₁₃-PLA₇-PCL₃, the yield of loading raised with the crosslinker content (MS1 and MS2) for each payload (FIG. 1). The introduction in the MS composition of a degradable tri-arm crosslinker instead of the linear one did not hinder the travoprost loading (MS7).

When the hydrophilic monomer m-PEGMA is replaced by another main monomer, for instance the tert-butyl methacrylate, the travoprost loading was still feasible with a high efficiency, higher than 90% (MS8).

For the more hydrophobic crosslinker, PEG₁₃-PCL₈, the efficacy of travoprost loading increased with the crosslinker content, from 5% (MS3) up to 50% (MS6) (FIG. 1). An efficient loading of travoprost was also obtained with a more hydrophobic crosslinker, PEG₂-PCL₁₂ at a content of 30% (MS9). A high degree of polymer crosslinking did not hinder an efficient drug loading on the degradable microspheres.

Compared to the concentration of travoprost in the eye drops solution used in glaucoma treatment at 40 μg/mL (Travartan*), significant amounts of travoprost (=1 or =2 mg/mL) corresponding to 25-fold or 50-fold the eye drop concentration were loaded on degradable microspheres after their synthesis according to a simple process of mixing. The microspheres of example 1 concentrate efficiently the travoprost molecules.

### Example 3. Study of the In Vitro Release of Travoprost from Loading Microsphere According to Example 2

After drug loading and freeze drying as described in example 2 the swelling step of microspheres was performed for 10 min in 10 mL of 0.9% NaCl saline solution. After the removal of saline, 50 mL of PBS (Sigma P-5368; 10 mM phosphate buffered saline; NaCl 0,136 M; KCl 0,0027 M; pH 7.4) were added. Drug elution occurred at 37° C. under shaking (150 rpm), the tubes were placed horizontally in the oven. Samples (1 mL) were withdrawn after 2 h, 24 h and every 3 or 4 days for 25 days. At each sampling time, the medium was completely renewed with fresh PBS. The amounts of free travoprost in saline and PBS supernatants were determined by RP-HPLC at 222 nm on a C₁₈ column (46×150 mm) using a mobile phase made of acetonitrile/water containing 0.1% TFA (60:40, v/v) at a flow rate of 1 mL/min in the isocratic mode at 25° C.

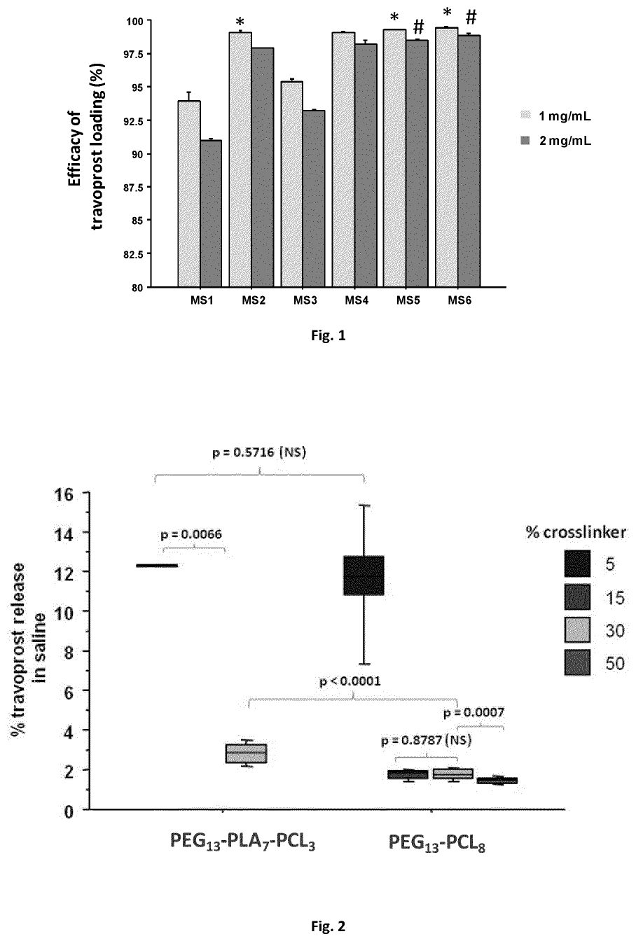

The effect of MS composition in terms of travoprost elution during the hydration of travoprost loaded MS in saline is described in FIGS. 2 & 3.

During the swelling of the loaded microspheres for the two travoprost payloads, the hydrophobicity of crosslinker had no effect on the drug release for MS at 5% of crosslinker (MS1 vs MS3). A significant but faint effect of the crosslinker composition was observed for microspheres at 30% crosslinker (MS2 vs MS5). The main parameter that controls the drug release during the hydration of travoprost-loaded MS is the concentration of each crosslinker for the two drug payloads. The travoprost release in saline was significantly reduced at crosslinker concentration higher than 5%. For the microspheres at high crosslinker concentration (15-30-50 mol %) the amount of travoprost eluted during the hydration step was similar, around 2%.

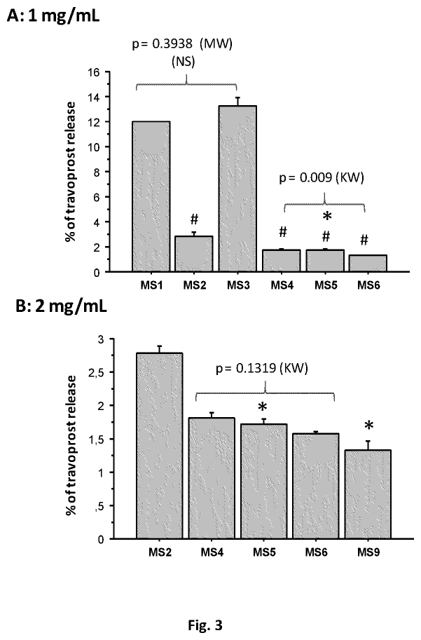

For each payload, the effect of crosslinker composition and concentration on travoprost release is given in FIG. 3. For the two payloads, the drug release was reduced for highly crosslinked MS at 15-30-50 mol % of crosslinkers. The level of travoprost loading had few effects on travoprost release during the MS swelling in saline. For the payload at 2 mg/mL, the drug elution in saline decreased significantly with the hydrophobicity of the crosslinker for MS at 30 mol % of each crosslinker (PEG₁₃-PLA-PCL₃, PEG₁₃-PCL₈ and PEG₂-PCL₁₂).

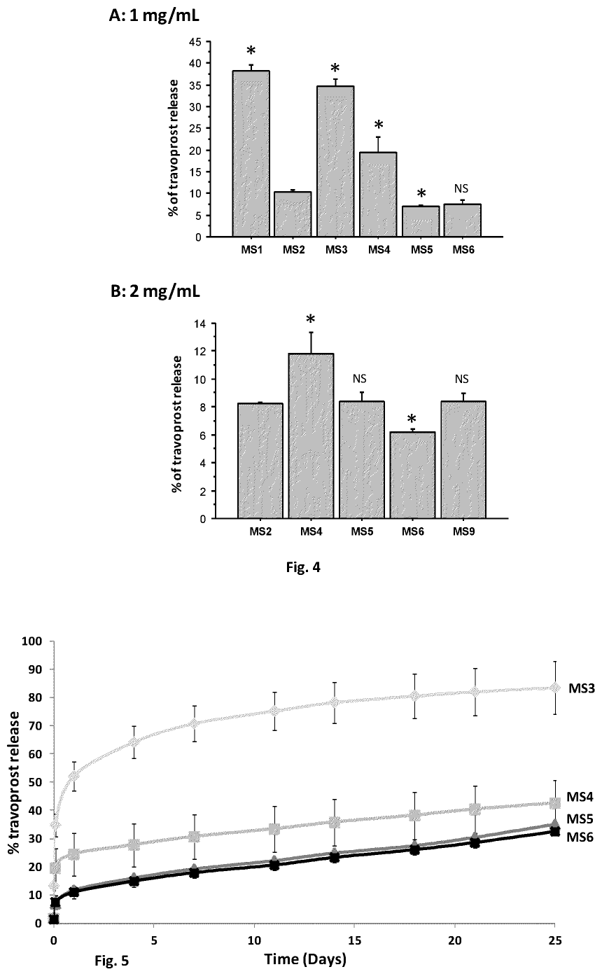

Then, after the swelling step in saline, the microspheres were transferred in PBS for travoprost release at 37° C. Drug elution after 2 h of incubation of the loaded microspheres in a saline buffered medium at pH 7.4 was shown in FIG. 4.

The passage of the travoprost loaded MS to the PBS triggered an important drug release for MS at 5 mol % of crosslinker (MS1 and MS3): =35% of drug release in 2 h. Travoprost elution in PBS was reduced at around 10% for the highly crosslinked microspheres at 30 mol % of each degradable crosslinkers, i.e. PEG₁₃-PLA₇-PCL₃, PEG₁₃-PCL₈ and PEG₂-PCL₁₂. Travoprost elution from the microspheres at 30% of crosslinker (MS2, MS5, MS9) was nearly the same (FIG. 4). A lower release was obtained with MS6 at 50 mol % of the crosslinker PEG₁₃-PCL₈. The degree of MS crosslinking controls the travoprost release during the first hours of incubation in PBS as observed previously during the swelling in saline.

The results of travoprost release after the hydration step and during the incubation in PBS are summarized in Table 3 for the drug loading at 1 mg/mL and in Table 4 for the payload at 2 mg/mL.

TABLE 3
Travoprost elution after swelling in saline (0.9% NaCl) for MS loaded at 1
mg/mL and after their subsequent transfer in PBS according to example 3.

| | % of travoprost release | | | | MS in vitro |
|---|---|---|---|---|---|
| Test | During MS | After 1 day | After 7 days | After 18 | degradation |
| number | swelling | in PBS | in PBS | days in PBS | time (days) |
| MS1 | 11.9 ± 0.06 | 49.11 ± 2.60 | 61.4 ± 2.29 | 65.4 ± 2.3 | 12 |
| MS2 | 2.82 ± 0.73 | 14.6 ± 0.74 | 22.4 ± 0.29 | 32.7 ± 0.42 | >180 |
| MS3 | 13.2 ± 1.66^{# (NS)} | 52.1 ± 5^{# (NS)} | 70.7 ± 6.3^{# (NS)} | 80.5 ± 7.8^{#} ^{(NS)} | 180 |
| MS4 | 1.71 ± 0.3 | 24.3 ± 7.5 | 30.6 ± 7.8 | 38.1 ± 8.37 | >180 |
| MS5 | 1.74 ± 0.19 | 11.5 ± 1.3 | 19.08 ± 1.24 | 27.4 ± 1.4 | >>180 |
| MS6 | 1.32 ± 0.11 | 10.9 ± 2.1 | 17.8 ± 1.85 | 26 ± 1.78 | >>>180 |
| MS8 | 0.52 ± 0.1 | 4.3 ± 1.6 | 7.7 ± 1.4 | 11.6 ± 1.4 | >180 |
| MW* | p = 0.0209 | p = 0.0209 | p = 0.0209 | p = 0.0209 | — |
| KW** | p = 0.0005 | p = 0.0006 | p = 0.0005 | p = 0.0005 | — |

*The non-parametric Mann-Whitney test (MW) was used to compare the effect of crosslinker PEG₁₃-PLA₇-PCL₃ content (5 mol % or 30 mol %) in MS1 and MS2, respectively, on travoprost release and to compare the effect of crosslinker composition at 5 mol % between MS1 and MS3 ^{#}.
**KW: the non-parametric kurskall-Wallis test was used to compare the effect of crosslinker PEG13-PCLg content (5 mol %, 15 mol %, 30 mol % and 50 mol % for MS3, MS4, MS5 and MS6, respectively) on travoprost release. The significance was set at p < 0.05.
Data are means.
NS: not significant.

For the travoprost payload at 1 mg/mL, the effect of crosslinker composition (PEG₁₃-PLA₇-PCL₃ or PEG₁₃-PCL₈) at 5 mol % had no effect on travoprost elution in PBS (Table 3). On the contrary, increasing the concentration of the two crosslinkers in MS significantly reduced the release of travoprost in the PBS at the different time points. At 15 mol %, 30 mol % and 50 mol % of crosslinker, the flow rate of travoprost in PBS was reduced compared to MS at 5 mol % of crosslinker. Replacement of the hydrophilic m-PEGMA with the tert-butyl methacrylate monomer (MS8) led to a slow down of travoprost release in PBS.

For the travoprost payload at 2 mg/mL, the increasing of crosslinker PEG₁₃-PCL₈ content in MS4, MS5 and MS6 (15 mol %, 30 mol %, 50 mol %) reduced significantly the release of travoprost in PBS after 1 day, 1 week and 18 days. On the other hand, MS7 at 5 mol % of the tri-arm crosslinker PEG₁₃-PLA₇-PCL₃ released rapidly the travoprost molecules as observed for MS1 at 5 mol % of the linear crosslinker (Table 4).

TABLE 4
Travoprost elution after swelling of MS loaded at 2 mg/mL in saline (0.9%
NaCl) and after their subsequent transfer in PBS according to example 3.

| | | | | | MS |
|---|---|---|---|---|---|
| | % of travoprost release | | | | in vitro |
| Test | During MS | After 1 day | After 7 days | After 18 days | degradation |
| number | swelling | in PBS | in PBS | in PBS | time (days) |
| MS2 | 2.70 ± 0.29 | 12.74 ± 0.26 | 21.2 ± 0.27 | 33.1 ± 0.68 | >180 |
| MS4 | 1.8 ± 0.15 | 15.7 ± 3.9 | 22.3 ± 4.53 | 30.4 ± 5.19 | >180 |
| MS5 | 1.7 ± 0.27 | 16.05 ± 5.4 | 23 ± 4.58 | 31.4 ± 3.56 | >>180 |
| MS6 | 1.5 ± 0.11 | 9.7 ± 0.77 | 15.7 ± 1.84 | 23.3 ± 2.63 | >>>180 |
| MS7 | 6.4 ± 0.28 | 36.4 ± 0.99 | 55.4 ± 1.05 | 68.3 ± 0.75 | ≈30 |
| MS9 | 1.3 ± 0.28 | 13.1 ± 0.9 | 22.7 ± 1.32 | 34.1 ± 2.01 | >>>180 |
| KW* | p = 0.1319 | p = 0.0025 | p = 0.0018 | p = 0.0047 | — |
| | (NS) | | | | |
| KW** | p = 0.003 | p = 0.9915 | p = 0.4431 | p = 0.5469 (NS) | — |
| | | (NS) | (NS) | | |

*The non-parametric kurskall-Wallis test was used to compare the effect of the PEG₁₃-PCL₈ crosslinker content (15 mol %, 30 mol % and 50 mol % for MS4, MS5 and MS6, respectively) on travoprost release.
**Non-parametric kurskall-Wallis test (KW) was used to compare the effect of the crosslinkers composition (PEG₁₃-PLA₇-PCL₃, PEG₁₃-PCL₈, PEG₂-PCL₁₂) at 30 mol % on travoprost release from MS2, MS5 and MS9. The significance was set at p < 0.05.
NS: non-significant.
The data are means.

At 30 mol % of crosslinker, the travoprost release in PBS was not significantly different between the 3 batches of microspheres MS2, MS5 and MS9. These results confirmed that the hydrophobicity of the crosslinkers has a low effect on the control of the release of travoprost, unlike the degree of microsphere crosslinking.

The effect of the crosslinker PEG₁₃-PCL₈ content in MS on travoprost release is summarized in FIG. 5 and in FIG. 6.

Compared to microsphere at 5 mol % of crosslinker which quickly released travoprost, the drug release was less important for the highly crosslinked microspheres (15 mol %, 30 mol %, 50 mol %) showing an effect of hydrogel mesh size on the drug release for a drug loading target of 1 mg/mL, (FIG. 5).

For the payload at 2 mg/mL, MS at 15 or 30 mol % of crosslinker (PEG₁₃-PLA-PCL₃, PEG₁₃-PCL₈, PEG₂-PCL₁₂) released travoprost at a similar flow rate during 1 month in PBS (FIG. 6). MS6 at 50 mol % of crosslinker PEG₁₃-PCL₈ provided a lower delivery of travoprost compared to the MS at 15 mol % or 30 mol % of crosslinker. As conclusion, a crosslinker concentration between 15 mol % and 50 mol % allows to control the burst after hydration of the microspheres and then the elution rate over time of the travoprost to get a controlled and sustained release.

### Example 4. Extemporaneous Loading of Travoprost on Sterile Microspheres According to Example 1

After the sieving step, a suspension of 250 μL of microspheres in 15 mL of a solution containing 2.5% (w/v) of mannitol was prepared. After homogenization, the pellet of microspheres was recovered, frozen-dried and sterilized by e-beam radiation (15-25 kilograys).

Then, 500 μL or 1 mL of water were added, before the addition of 500 μL or 1 mL, respectively, of travoprost solution (Sigma, PHR 1622-3 mL lot LRAA5292 (0.499 μg/mL in water/acetonitrile (70/30)).

The loading step was done at room temperature for 5 min under stirring on a tube rotator (=30 rpm). Then, the supernatants were removed for the measurement of unbound travoprost by fluorimetry (λeₑₓ 220 nm, λ_{Em} 310 nm). The amount of travoprost was obtained by extrapolation from a standard curve (0.6 to 20 μg/mL).

Table 5 summarizes extemporaneous travoprost loading on dry and sterile MS of different formulations tested.

TABLE 5
Travoprost loading in the microspheres
according to example 3

| Test | Travoprost loading (mg/mL) for 0.5 mL of |
|---|---|
| number | travoprost solution (% of loading efficacy) |
| MS1 | 0.9 ± 0.011 |
| | (90%) |
| MS5 | 1.98 ± 0.14 |
| | (99%) |

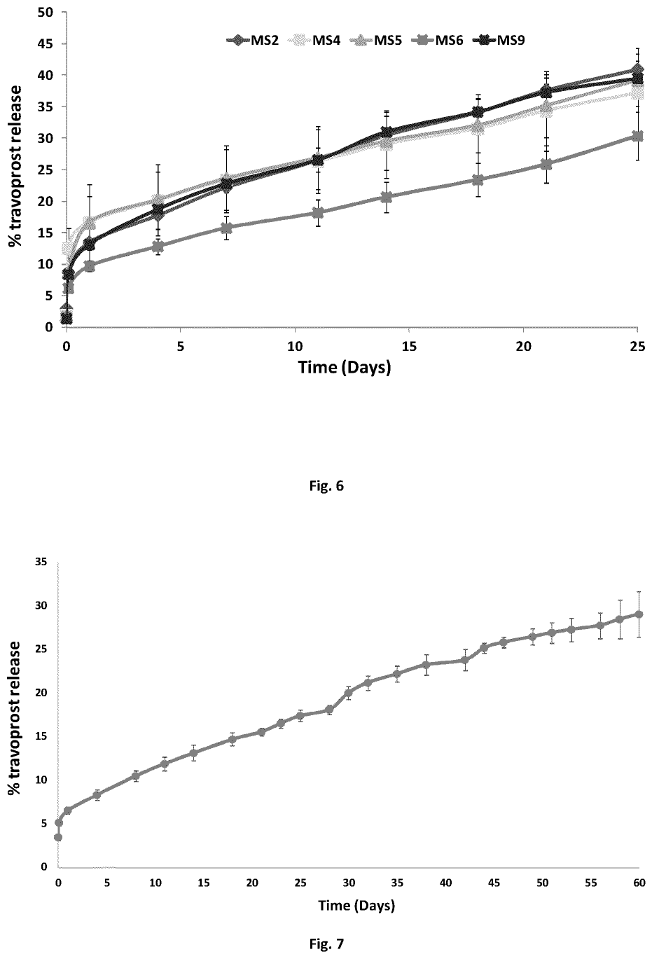

Then, PBS (50 mL) was added to the microspheres loaded with travoprost for the in vitro drug release experiment (37° C., 150 rpm). The medium was completely renewed after each sample collection. The amounts of travoprost in PBS supernatants were determined by RP-HPLC at 222 nm on a C₁₈ column (46×150 mm) using a mobile phase made of acetonitrile/water containing 0.1% TFA (60:40, v/v) at a flow rate of 1 mL/min in the isocratic mode at 25° C. The in vitro release from MS5 is shown in FIG. 7.

After extemporaneous loading on dry and sterile MS, the initial burst release of travoprost was slow (3.5%) and sustained for at least two months (FIG. 7).

### Example 5. Extemporaneous Loading of Other Prostaglandin Analogues on Sterile Microspheres According to Example 1

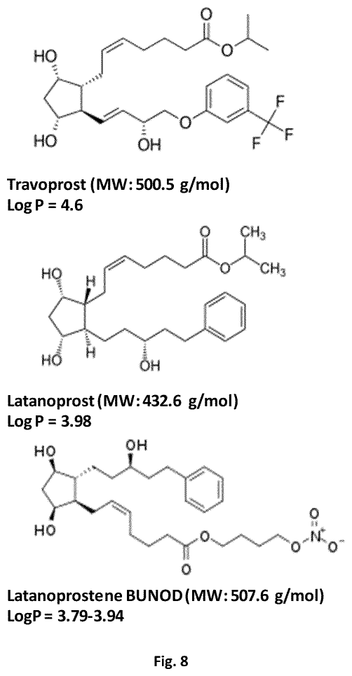

After the sieving step of MS5, 100 μL or 250 μL of pellets were suspended in 5 mL of a solution containing 2.5% (w/v) of mannitol. After homogenization, the pellets of microspheres were recovered, frozen-dried and sterilized by e-beam radiation (25 kilograys). The structures of prostaglandin analogues used for drug loading trials are displayed in FIG. 8.

Latanoprost Loading To the dry and sterile pellets of microspheres (250 μL), 500 μL of latanoprost solution (TRC-L177280-10MG) at 2 mg/mL in acetonitrile/water mixture (70/30) were added. After 5 min of mixing at room temperature under stirring on a tube rotator (=30 rpm), the supernatants were removed for the measurement of unbound latanoprost by RP-HPLC at 210 nm on a C₁₈ column (46×150 mm) using a mobile phase made of acetonitrile/water containing 0.1% TFA (60:40, v/v) at a flow rate of 1 mL/min in the isocratic mode at 25° C. The amount of latanoprost in supernatants was obtained by extrapolation from a standard curve (0.5 to 20 μg/mL). The loaded dose was calculated by subtracting the final amount of latanoprost from the initial amount. The latanoprost loading for 1 mL of beads was obtained by multiplying by 4 the quantity loaded on 250 μL of MS.

### Latanoprostene BUNOD Loading

Latanoprostene BUNOD is a nitric oxide (NO)-donating prostaglandin F2a analogue approved for the reduction of intraocular pressure in patients with open-angle glaucoma or ocular hypertension. To the dry and sterile pellets of microspheres (100 μL), 700 μL of latanoprotene BUNOD (TRC-L177335-2.5MG) solution at 1 mg/mL in acetonitrile/water mixture (70/30%) were added. After 5 min of mixing at room temperature under stirring on a tube rotator (=30 rpm), the supernatants were removed for the measurement of unbound drug by RP-HPLC at 210 nm on a C₁₈ column (46×150 mm) using a mobile phase made of acetonitrile/water containing 0.1% TFA (60:40, v/v) at a flow rate of 1 mL/min in the isocratic mode at 25° C. The amount of latanoprotene BUNOD in supernatants was obtained by extrapolation from a standard curve (0.25 to 25 μg/mL). The loaded dose was calculated by subtracting the final amount of latanoprotene BUNOD from the initial amount. The latanoprotene BUNOD loading for 1 mL of beads was obtained by multiplying by 10 the quantity loaded on 100 μL of MS.

The loading values of latanoprost and latanoprostene BUNOD on dry and sterile pellets of MS5 are summarized on table 6. Latanoprost and travoprost are potent anti-glaucoma drugs efficient at low dose (40-50 μg/mL). On contrary, latanoprostene BUNOD is efficient at higher concentration (240 μg/mL), which implies obtaining a higher drug payload for a sustained release of therapeutic doses for several weeks following a single subconjunctival injection.

TABLE 6
Extemporaneous loading of prostaglandin analogues on MS5 after freeze
drying and e-beam sterilization of degradable microspheres.

| | Prostaglandin analogues | |
|---|---|---|
| | loading on MS5 (mg/mL) | |
| | Latanoprost | Latanoprostene BUNOD |
| Glaucoma eye drops concentration | 50 μg/mL | 240 μg/mL |
| Partition coefficient P (Log P) of the | 3.98 | 3.79-3.94 |
| prostaglandin analogues* | | |
| Drug loading on MS5 | 3.83 ± 0.07 | 6.58 ± 0.1 |
| (mg/mL) | (98%)** | (94%) |

*https://pubchem.ncbi.nlm.nih.gov/.
**loading efficiencies (%) were calculated by the following equation ((Drug in feed-Drug in supernantant after the loading step)/Drug in feed) × 100.
Data are means.

The loading of each prostaglandin analogue on the sterile MS5 batch was efficient (yield>90%) after a short period of mixing (5 min at room temperature). The payload of degradable MS5 with prostaglandin analogues was important, since at least 6.5 mg of latanoprostene BUNOD were loaded on the degradable microsphere. This rapid loading confirms the affinity that exists between degradable polymers formulated in microspheres of example 1 and some of the prostaglandin analogues used for glaucoma treatment.

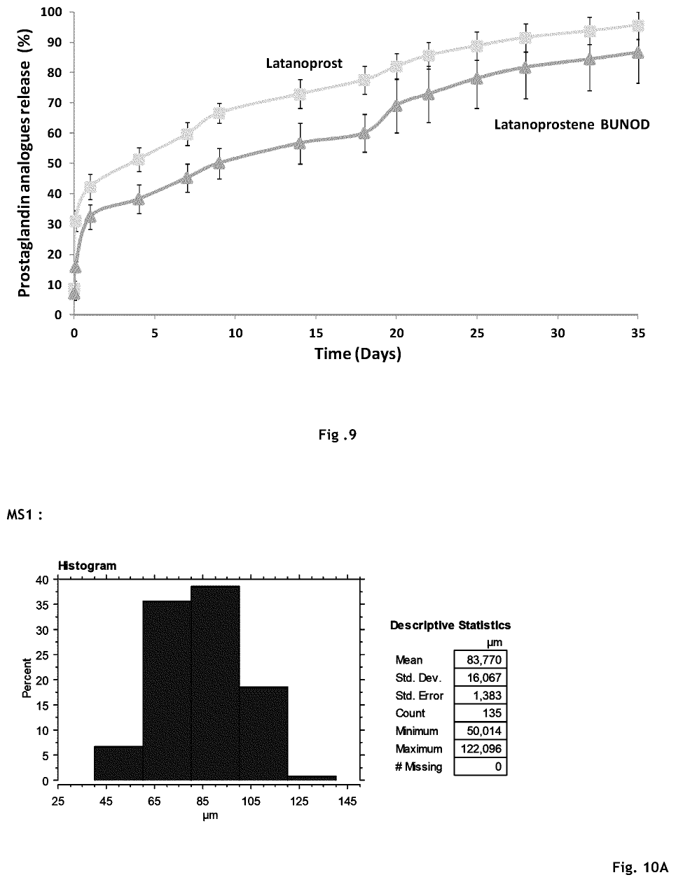

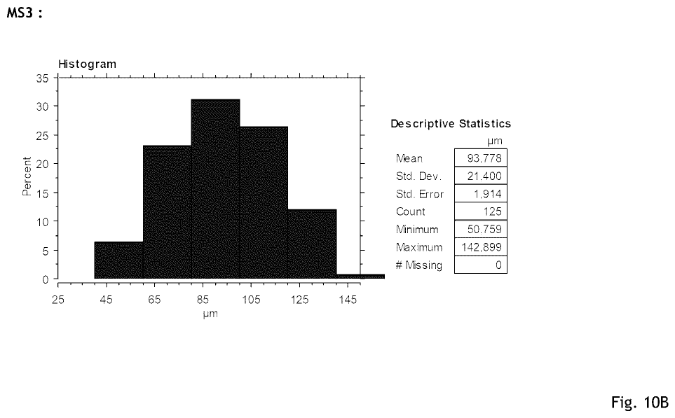

Then, immediately after the loading step, 50 mL of PBS (Sigma P-5368; 10 mM phosphate buffered saline; NaCl 0,136 M; KCl 0,0027 M; pH 7.4) was added to the microspheres loaded with latanoprost or latanoprostene BUNOD for the in vitro drug release (37° C., 150 rpm). The medium was completely renewed after each sample collection (10 min, 2 h, 24 h and every 3-4 days during 5 weeks). The amounts of prostaglandin analogues in PBS supernatants were determined by RP-HPLC at 210 nm for latanoprost and latanoprostene BUNOD on a C₁₈ column (46×150 mm) using a mobile phase made of acetonitrile/water containing 0.1% TFA (60:40, v/v) at a flow rate of 1 mL/min in the isocratic mode at 25° C. The results are summarized in Table 7 and in FIG. 9 for each drug.

TABLE 7
Latanoprost and latanoprostene BUNOD elution in PBS after the
extemporaneous loading on sterile MS5.

| | % of prostaglandin analogues release | | | |
|---|---|---|---|---|
| Prostaglandin | After 10 | After 1 day | After 7 days | After 18 days |
| analogues | min in PBS | in PBS | in PBS | in PBS |
| Latanoprost | 8.7 ± 0.5 | 42.3 ± 10 | 59.7 ± 11.1 | 77.5 ± 11.9 |
| Latanoprostene BUNOD | 7.04 ± 2.2 | 32.4 ± 4.1 | 45.2 ± 4.5 | 60 ± 6.2 |

The data are means.

The in vitro elution experiments show that is possible after a rapid extemporaneous loading on sterile MS5 to achieve a delivery of prostaglandin analogues for 5 weeks in vitro, the intensity of drug release depends on the prostaglandin analogue. During the first 10 min of incubation in PBS, the release of the two drugs was similar (Table 7), but after 24 h in PBS, the values of drug release were different between the two drugs, elution of latanoprost was faster than that of latanoprostene BUNOD. Elution of latanosprost and latanoprostene BUNOD are practically complete after 5 weeks of incubation (FIG. 9), while elution of travoprost occurred at lower flow rate (FIG. 7).

The interaction between the prostaglandin analogues and the degradable microspheres varies according to the structure of the drug. The degree of microsphere crosslinking is not the only parameter to be involved for a controlled and sustained drug delivery of a prostaglandin analogue for glaucoma treatment. The molecular weight of the drug or the presence of certain atoms such fluorine for the travoprost, or nitrogen for latanoprostene BUNOD, could have an effect on the affinity of the molecule for the crosslinked polymer.

In vitro, the release of prostaglandin analogues from a DDS can be accelerated compared to the in vivo conditions as shown previously (Natarajan et al., 2014. ACS Nano. 8: 419-29). For liposomes containing latanoprost, a plateau at 60% of the drug released was obtained in 30 days in PBS, while after a single subconjunctival injection of the liposomes in non-human primates, a reduction of intraocular pressure was measured during 120 days. As the main explanation, the authors suppose that the release of latanoprost from the liposomes was slower in the eye, due to the lower volume of liquid in the subconjunctival space, where the sink condition can no longer be applied, which slows down the drug release. In another hypothesis, the authors assume that the released latanoprost from the liposomes was not cleared fast from the eye, the ocular residence time would be increased.

These observations suggest that the prostaglandin analogues loaded on the microspheres of the example 1 would elute in vivo long enough after a single subconjunctival injection to reduce intraocular pressure for several months.

### Example 6: Size Distribution of Sterile Hydrophilic Microspheres (MS1 Et MS3) after Swelling in Saline

The sterile microspheres from two batches with crosslinker having different hydrophobicity have similar size distribution. No difference in terms of size was found according to the Mann-Whitney non-parametric test (p=0.2493). (See FIGS. 10A and 10B).

1. A composition comprising an effective amount of a prostaglandin analogue, at least one hydrophilic degradable microsphere comprising a crosslinked matrix, and a pharmaceutically acceptable carrier for administration by injection, the crosslinked matrix being based on at least:
a) from 10 to 90 mol % of hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 to 30 mol % of a cyclic monomer of formula (II): 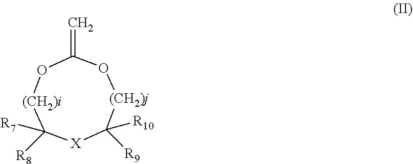
wherein:
R₇, R₈, R₉ and R₁₀ are, independently of one another, hydrogen atom, a (C₁-C₆)alkyl group or an aryl group;
i and j are independently of one another an integer chosen between 0 and 2; and
X is a single bond or an oxygen atom; and
c) from 5% to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ—PEGₚ-Xₖ—CO—(CR₁₁═CH₂) (IIIa);
W(PEGₚ-Xₙ—O—CO—(CR₁₁═CH₂))_{z} (IIIc);
wherein:
each Rₙ is independently of one another hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z represents the number of arms of the PEG molecule and is an integer from 3 to 8;
wherein mol % of components a) to c) are expressed relative to the total number of moles of compounds a), b) and c).

2. The composition of claim 1, wherein the degradable block copolymer cross-linker c) is selected from the group consisting of compounds of general formula (IIIa) or (IIIc), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

3. The composition of claim 1, wherein the degradable block copolymer cross-linker c) is of general formula (IIIa) or (IIIIc), wherein X represents PCL or PLAPCL.

4. The composition of claim 1, wherein the degradable block copolymer cross-linker c) is present in the reaction mixture in an amount of between 5 mol % and 60 mol %, relative to the total number of moles of the monomers.

5. The composition of claim 1, wherein the cyclic monomer b) is selected from the group consisting of 2-methylene-1,3-dioxolane, 2-methylene-1,3-dioxane, 2-methylene-1,3-dioxepane, 2-methylene-4-phenyl-1,3-dioxolane, 2-methylene-1,3,6-trioxocane and 5,6-benzo-2-methylene-1,3-dioxepane.

6. The composition of claim 1, wherein the hydrophilic monomer a) is selected from the group consisting of sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate.

7. The composition of claim 1, wherein the crosslinked matrix of the hydrophilic degradable microsphere is further based on a chain transfer agent d).

8. The composition of claim 1, comprising between 1 and 6 mg/mL of a prostaglandin analogue.

9. The composition of claim 1 wherein the block copolymer cross-linker c) is a compound of general formula (IIIc) wherein p is 7, X=PLAPCL, n=10 and z is 3.

10. A method for preventing and/or treating ocular hypertension or glaucoma, which comprises the step of administering a composition as defined in claim 1 to a subject in need thereof.

11. The method according to claim 10, wherein the prostaglandin analogue is released during a period of at least 3 weeks.

12. A hydrophilic degradable microsphere for use for delivering an effective amount of a prostaglandin analogue, to a subject in need thereof, the hydrophilic degradable microsphere comprising a crosslinked matrix, the crosslinked matrix being based on at least:
a) from 10 to 90 mol % of hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ CH₃, —(CR₂R₃)ₘ—OH or —(CH₂)ₘ—NR₅R₆ with m being an integer from 1 to 30;
R₁, R₂, R₃, R₄, R₅ and R₆ are, independently of one another, hydrogen atom or a (C₁-C₆)alkyl group;
b) from 0.1 to 30 mol % of a cyclic monomer of formula (II): 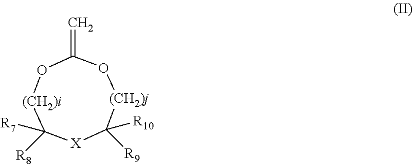
wherein:
R₇, R₈, R₉ and R₁₀ are, independently of one another, hydrogen atom, a (C₁-C₆)alkyl group or an aryl group;
i and j are independently of one another an integer chosen between 0 and 2; and
X is a single bond or an oxygen atom; and
c) from 5% to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ—PEGₚ-Xₖ—CO—(CR₁₁═CH₂) (IIIa):
W(PEGₚ-Xₙ—O—CO—(CR₁₁═CH₂))_{z} (IIIc);
wherein:
each R₁₁ is independently of one another hydrogen atom or a (C₁-C₆)alkyl group;
X is independently PLA, PGA, PLGA, PCL or PLAPCL:
n and k are independently integers from 1 to 150;
p is an integer from 1 to 100:
W is a carbon atom, a C₁-C₆-alkyl group or an ether group comprising 1 to 6 carbon atoms;
z represents the number of arms of the PEG molecule and is an integer from 3 to 8:
wherein mol % of components a) to c) are expressed relative to the total number of moles of compounds a), b) and c).

13. The hydrophilic degradable microsphere according to claim 12, wherein the prostaglandin analogue, is released during a period of at least 3 weeks.

14. Pharmaceutical kit comprising:
i) at least one hydrophilic degradable microsphere in association with a pharmaceutically acceptable carrier for administration by injection;
ii) an effective amount of a prostaglandin analogue, and
iii) optionally an injection device,
the hydrophilic degradable microsphere and the prostaglandin analogue being packed separately, and wherein the hydrophilic degradable microsphere comprises a crosslinked matrix, the crosslinked matrix being based on at least:
a) from 10 to 90 mol % of hydrophilic monomer of general formula (I):
(CH₂═CR₁)—CO-D (I)
wherein:
D is O—Z or NH—Z, with Z being —(CR₂R₃)ₘ—CH₃, —(CH₂—CH₂—O)ₘ—H, (CH₂—CH₂—O)ₘ CH₃, —(CR₂R₃)ₘ—OH or —(CH₂)ₘ—NR₅R₆ with m being an integer from 1 to 30:
R₁, R₂, R₃, R₄, R₅ and R₆ are, independently of one another, hydrogen atom or a (C₁-C₆)alkyl group;
b) from 0.1 to 30 mol % of a cyclic monomer of formula (II): 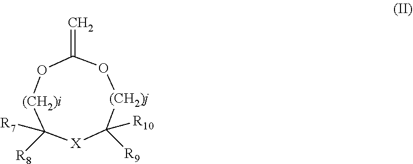
wherein:
R₇, R₈, R₉ and R₁₀ are, independently of one another, hydrogen atom, a (C₁-C₆)alkyl group or an aryl group;
i and j are independently of one another an integer chosen between 0 and 2; and
X is a single bond or an oxygen atom; and
c) from 5% to 90 mol % of a linear or star-shaped degradable block copolymer cross-linker having a partition coefficient P of between 0.50 and 11.20, or a hydrophobic/hydrophilic balance R between 1 and 20, said degradable block copolymer cross-linker having the formula:
(CH₂═CR₁₁)—CO—Xₙ—PEGₚ-Xₖ—CO—(CR₁₁═CH₂) (IIIa):
W(PEGₚ-Xₙ—O—CO—(CR₁₁═CH₂))_{z} (IIIc);
wherein:
each R₁₁ is independently of one another hydrogen atom or a (C₁-C₆)alkyl group;
X is independently PLA, PGA, PLGA, PCL or PLAPCL;
n and k are independently integers from 1 to 150;
p is an integer from 1 to 100;
W is a carbon atom, a C₁-C₆-alkyl group or an ether group comprising 1 to 6 carbon atoms;
z represents the number of arms of the PEG molecule and is an integer from 3 to 8:
wherein mol % of components a) to c) are expressed relative to the total number of moles of compounds a), b) and c).

15. The composition of claim 1, wherein the degradable block copolymer cross-linker c) is selected from the group consisting of compounds of general formula (IIIa), wherein:
X=PLA, n+k=12 and p=13; or
X=PLAPCL, n+k=10 and p=13; or
X=PLAPCL, n+k=9 and p=13; or
X=PLAPCL, n+k=8 and p=13; or
X=PCL; n+k=8 and p=13; or
X=PLGA; n+k=12 and p=13; or
X=PCL, n+k=10 and p=4; or
X=PCL, n+k=12 and p=2.

16. The composition of claim 1, wherein the degradable block copolymer cross-linker c) is present in the reaction mixture in an amount of between 15% and 60% by mole, relative to the total number of moles of the monomers.

17. The composition of claim 1, wherein the prostaglandin analogue is travoprost.

18. The method of claim 10, wherein the prostaglandin analogue is travoprost.
